(19) United States
(12) Patent Application Publication
(10) Pub. No.: US 2014/0296146 A1
(43) Pub. Date: Oct. 2, 2014

(54) Thermostable Inhibitors of Activation of the Blood Clotting System Through Contact with Foreign Surfaces

(71) Applicants: Synapse B.V., Maastricht (NL); Universiteit Maastricht, Maastricht (NL)

(72) Inventors: Tilman Mathias Hackeng, Cadier en Keer (NL); Dennis Peter Leonardo Suijlen, Nieuwstadt (NL); Hendrik Coenraad Hemker, Maastricht (NL); Rafael Jesus Apitz-Castro, San Bernardino (VE)

(73) Assignees: Synapse B.V., Maastricht (NL); Universiteit Maastricht, Maastricht (NL)

(21) Appl. No.: 14/240,292

(22) PCT Filed: Aug. 20, 2012
(86) PCT No.: PCT/NL2012/050580

§ 371 Date: Feb. 21, 2014

Related U.S. Application Data
(60) Provisional application No. 61/526,368, filed on Aug. 23, 2011.

(30) Foreign Application Priority Data
Aug. 23, 2011 (EP) 11178517.6

(51) Int. Cl. C07K 14/745

(52) U.S. Cl. CPC C07K 14/745
USPC 514/13.7; 536/23.5; 530/324; 435/13

(57) ABSTRACT

The present invention relates to the field of blood clotting. Specifically, the invention relates to particular inhibitors of artificial activation of the blood clotting process through contact with foreign surfaces.

### FIELD OF THE INVENTION

The present invention relates to the field of blood clotting. Specifically, the invention relates to particular inhibitors of artificial activation of the blood clotting process through contact with foreign surfaces.

### BACKGROUND OF THE INVENTION

The clotting system of the blood is essential for stopping bleeding but also is instrumental in the development of thrombosis. Equally many people die from bleeding or thrombosis as from cancer. To be able to detect over-activity of the clotting system (that fosters thrombosis) or under-activity (which causes a bleeding tendency), therefore is of paramount medical importance. Such function testing is an ex vivo laboratory procedure that has to be executed on blood taken from a blood vessel via a needle into a vial. This necessarily implies contact of the blood with “foreign” surfaces, i.e. surfaces other than the normal inside of the blood vessels.

As soon as the blood comes into contact with such surfaces the clotting process is set into motion. Blood clotting factor XII (FXII) or Hageman factor adsorbs onto the foreign surface and thereby turns into an active enzyme that, with the help of other plasma proteins (high molecular weight kininogen and prekallikrein) activates factor XI, which in its turn activates factor IX. If care is taken to take the blood into a solution that contains a substance that binds free Ca²⁺ ions (e.g. Na-citrate or EDTA) no further reaction takes place. FIXa can continue the process only if such ions are available.

Taking blood in citrate therefore is a standard procedure if blood is to be collected for the study of the clotting system. Nevertheless in such blood, factors XII, XI and IX are likely to be already activated to a greater or lesser extent.

The part of the thrombin generation process up to activation of FIX is called the contact activation system. The contact activation process as such is not a part of the natural thrombin generation system, but FXI and FIX do have a role in this natural system and need to remain non-activated if its function is to be probed correctly. Prior involuntary activation interferes with assessment of the natural thrombin generating capacity and with any other type of measurement of the blood clotting system in which factors XI and IX play a role or where, on the contrary, such role is to be excluded.

It therefore is necessary to inhibit the contact activation system. Because the contact activation process starts as soon as the blood is taken from the patient and meets a foreign surface, such as the inside of the needle but especially the inside of the blood collection tube. This is why the inhibitor has preferably to be added to the mixture in the tube in which the blood is collected. Such mixture will contain citrate or another Ca²⁺-chelating substance and any other addition that may be required.

By adding such inhibitor of contact activation the blood remains in its native state and the natural thrombin generation (TG) process and its mechanisms or a relevant part thereof can then be studied after recalcification.

For being tested, the entire thrombin generation process is best triggered in the same way as it is under natural circumstances in a wound or thrombus, i.e. by tissue factor. When measuring thrombin activity in a sample comprising a Ca²⁺ chelating substance, sufficient Ca²⁺ has to be added to enable the blood clotting cascade.

Tissue factor is a membrane protein that occurs on perivascular cells that are only exposed to blood when the vessel wall is damaged. It also occurs in inactive form (“encrypted”) in certain white blood cells. It can be “decrypted” when these cells are activated by processes that normally occur during bacterial infection, such as exposure to lipopolysaccharides or other products from contaminating bacteria. For this reason, and also because of general medical and hospital hygiene, it is necessary that the tubes on which blood is taken are sterile to avoid aforementioned processes that would severely influence outcome of laboratory coagulation tests. An inhibitor of the contact activation system therefore should remain active during sterilization of the containers used for taking blood.

Thrombin generation is an extremely complicated web of enzymatical reactions. Its backbone is a series of proenzyme-enzyme reactions in which each proenzyme, once activated, activates the next one (the “clotting cascade”).

The contact activation system is not a part of the natural cascade but the necessary inhibition of the contact activation system should not, as a side effect, inhibit any of the multiple reactions of the normal thrombin generation network, i.e. of thrombin generation as triggered by tissue factor.

Many of the reactions in this network occur at a phospholipid-solute (plasma) interface. Only phospholipids that are normally at the inside of a cell can serve this purpose. As a part of the clotting process of blood specific cells (blood platelets) turn their membranes inside-out so as to facilitate thrombin formation. Bacterial contamination can potentially cause blood platelets and white blood cells to expose procoagulant phospholipids, which is another way to disturb the natural clotting process. This is a second reason why an inhibitor of the contact activation system therefore should remain active during sterilization.

Corn trypsin inhibitor (CTI) from its natural source, is known for the purpose of inhibiting contact activation of blood (U.S. Pat. No. 6,403,381). CTI is heat labile and therefore has a short shelf-life and must be stored under special conditions. More important is that it cannot be sterilized without losing activity.

There is thus a need for a substance that inhibits contact activation and is heat resistant.

### DETAILED DESCRIPTION OF THE INVENTION

Surprisingly, it has now been demonstrated that specific thermostable peptides inhibit contact activation of blood, while not inhibiting thromboplastin induced thrombin generation, i.e. not impeding the normal clotting process.

Accordingly, in a first aspect the present invention provides a composition comprising at least one calcium chelating substance with blood clotting inhibition activity and further comprising a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met.

The blood clotting system is known to the person skilled in the art, In short it is a sequence of proenzyme-enzyme conversions that for the most part occur at interfaces and that are controlled by positive and negative feedback reactions. It is succinctly described in the introduction of this application and in more detail in “The blood coagulation cascade” by Schenone M, Furie BC and Furie B. Current Opinion in Haematology 2004, Vol 11(4) pages 272-277.

Since a thermostable peptide according to the invention inhibits contact activation of the blood clotting process, it can conveniently be used in any biological fluid, including bodily fluids, including blood, where contact activation of the blood clotting system may occur. Accordingly, in all the embodiments of the present invention where blood is specified, other bodily fluids, including cerebrospinal fluid transudates and exudates, and all blood products wherein blood clotting may occur, including plasma, are equally intended and to be construed as part of the invention. Blood may be freshly drawn blood, but may also be blood or a blood product such as plasma that has been stored.

A calcium-chelating substance with blood clotting inhibition activity is herein defined as any substance that binds free Ca²⁺ ions while having blood clotting inhibition activity. By the binding of free Ca²⁺, the blood clotting cascade is interrupted since factor IX cannot be activated in the absence of free Ca²⁺ ions. Calcium-chelating substances with blood clotting activity are well known in the art and include e.g. ethylene-diamine-tetraacetic-acid (EDTA), citrate or salts of citrate, including Na-citrate. Preferably, the calcium chelating substance with blood clotting inhibition activity is compatible with measuring thrombin activity, a preferred substance is Na-citrate.

A peptide is herein defined as a polymer of amino acids linked by chemical bonds, preferably peptide bonds. In principle the amino acids may be any naturally occurring or unnatural or uncoded amino acids, or a mix thereof. A dipeptide consisting of two amino acids is the smallest peptide possible. A peptide may comprise from two to multiple thousand amino acids, e.g. 2, 3, 4, 5, 6 7, 8, 9, 10, 20, 30, 40, 50, 100, 200, 500, 800, 100, 1200, 1500, 2000 etc. amino acids.

Inhibition is herein defined as a reduction in an activity. The reduction may be any reduction, e.g. 1-fold, 10-fold, 100-fold, 1000-fold reduction.

Contact activation is herein defined as activation of the blood clotting process by contact of the blood with any other surface than its natural environment such as the inner surface of a vein. Such foreign surface may e.g. be a blood collection device or a hollow needle. The blood clotting process is explained earlier herein. It follows that Inhibition of contact activation is when contact activation is reduced.

About 40 amino acids is herein defined as approximately 40 amino acids, i.e. 38, 39, 40, 41, or 42 amino acids, preferably 40 amino acids.

A position corresponding to amino acid position 5 in SEQ ID NO: 1 is herein defined as the position in a peptide according to the present invention when aligned to SEQ ID NO: 1 that corresponds to the position of the amino acid Arginine (R) at position 5 of SEQ ID NO: 5.

Identity in the expression “A peptide which has at least 70% sequence identity with a peptide as shown in SEQ ID NO: 1, SEQ ID NO: 2, SEQ ID NO: 3, or SEQ ID NO:” is defined as known in the art, as a relationship between two or more amino acid (peptide) sequences by comparing the sequences. In the art, the percentage of “identity” indicates the degree of sequence relatedness between amino acid as determined by the match between strings of such sequences. Preferably, the percentage of identity is determined by comparing the whole SEQ ID NO as identified herein. However, part of such sequence may also be used. In this context, “part” means at least 50%, 60%, 70%, 80%, 90% or 100% of its length. Preferably 100% is used.

Two amino acid sequences are considered “similar” if the polypeptides only differ in conserved amino acid substitutions. In determining the degree of amino acid similarity, the skilled person takes into account “conservative” amino acid substitutions. Conservative amino acid substitutions refer to the interchange of amino acids having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; and a group of amino acids having sulphur-containing side chains is cysteine and methionine. Preferred conservative amino acids substitution groups are: valine-leucine-isoleucine, phenylalanine-tyrosine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Asn to gln or his; Asp to glu; Cys to ser or ala; Gln to asn; Glu to asp; Gly to pro; His to asn or gln; Ile to leu or val; Leu to ile or val; Lys to arg; Asn to gln or glu; Met to leu or ile; Phe to met, leu or tyr; Ser to thr; Thr to ser; Trp to tyr; Tyr to trp or phe; and, Val to ile or leu. In the case of Arg, preferred conservative substitutions and analogs therefore are defined later herein.

“Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1):387 (1984)), BestFit and FASTA (Altschul, S. F. et al., J. Mol. Biol. 215:403-410 (1990). The BLAST 2.0 family of programs which can be used for database similarity searches includes: BLASTP for protein query sequences against protein database sequences. The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Another preferred method to determine sequence identity and similarity is by using the algorithm Needleman-Wunsch (Needleman, S. B. and Wunsch, C. D. (1970) J. Mol. Biol. 48, 443-453, Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The following website could be used: http://www.ebi.ac.uk/Tools/emboss/align/index.html. Definitions of the parameters used in this algorithm are found at the following website: http://emboss.sourceforge.net/docs/themes/AlignFormats.html#id. Preferably, using this algorithm, Gap_penalty is 10.0 and Extend_penalty is 0.5.

The amino acid Arg is herein defined as the amino acid Arginine (R) or an analog of the amino acid Arginine; preferably the amino acid Arginine (R). An analog of Arg is herein defined as the any analog of the amino acid Arginine (R), whether natural, unnatural and/or synthetic known to the person skilled in the art. A preferred Arginine analog is selected from the group consisting of: monomethylarginine, symmetric and asymmetric dimethylarginine, canaline, δ-guanidino α-amino butyric acid, homoarginine, canavanine, citrulline, conformationally restricted analogs of Arginine, amino boronic acid analogs of Arginine, and other basic amino acids or derivatives thereof. Preferably, an Arginine analog is not Lysine or a derivative thereof. A preferred position where this definition applies is the Arginine at a position corresponding to amino acid position 5 in SEQ ID NO: 1.

In the composition according to the invention, the calcium-chelating substance with blood clotting inhibition activity and the thermostable peptide which inhibits contact activation of the blood clotting system may be present in the composition in any concentration and ratio as long as the composition still retains the activities of both compounds when in the final concentration in the blood, plasma or other biological fluid as described earlier herein. Preferably, the calcium chelating substance with blood clotting inhibition activity such as sodium citrate is present in such concentration that results in a final concentration from about 11 mM to 13 mM, more preferably 11 mM. The thermostable peptide according to the invention which inhibits contact activation of the blood clotting system is preferably present in such concentration that results in a final concentration in blood of from about 5 μg/ml to 500 μg/ml, more preferably 10 μg/ml to 100 μg/ml. Preferably, when present in a sampling container, the calcium chelating substance with blood clotting inhibition activity such as sodium citrate is present in a stock solution of 3.2 or 3.8% in a sampling container resulting in a final concentration in blood from about 11 mM to 13 mM, more preferably 11 mM. Preferably, the thermostable peptide according to the invention which inhibits contact activation of the blood clotting system is present in a stock solution in the sampling tube resulting in a final concentration in blood of from about 10 μg/ml to 100 μg/ml. The stock solution may have any stock concentration, including 5, 10, 20-fold the preferred final concentration. Preferably, the stock solution has 10-fold the final concentration. The person skilled in the art will understand that when whole blood is drawn, the final concentration of the calcium chelating substance and of the thermostable peptide according to the invention will result in about twice these concentrations in plasma, depending upon the volume taken by red blood cells, which is around 50% of the total volume of blood. In all embodiments of the present invention, the final concentration of the thermostable peptide according to the invention which inhibits contact activation of the blood clotting system may any concentration as long as the peptide still retains its inhibition activity; preferably the final concentration is from about 5 μg/ml to 500 μg/ml, more preferably 10 μg/ml to 100 μg/ml.

In all the embodiments of the present invention, the thermostable peptide according to the invention which inhibits contact activation of the blood clotting system may be any peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met. The consecutive sequence of at most about 40 amino acids comprising at least two or at least four cysteines, the amino acid Arg or an analog of Arg on a position corresponding to amino acid position 5 in SEQ ID NO: 1 and preferably at least the consecutive amino acid sequence Ile-Leu-Met may be 20, 21, 22, 23, 24, 25, 26, 27, 28, 29, 30, 31, 32, 33, 34, 35, 36, 37, 38, 39, 40, 41, 42 amino acids; preferably about 25-35 amino acids, more preferably about 27-32 amino acids, and most preferably about 29 amino acids. The consecutive sequence of at most about 40 amino acids comprising at least two or at least four cysteines, the amino acid Arg or an analog of Arg on a position corresponding to amino acid position 5 in SEQ ID NO: 1 and preferably at least the consecutive amino acid sequence Ile-Leu-Met, preferably comprises a pair of cysteines (i.e. totals two cysteines) that under physiological circumstances can form disulfide bridges. The consecutive sequence of at most about 40 amino acids comprising at least two or at least four cysteines, the amino acid Arg or an analog of Arg on a position corresponding to amino acid position 5 in SEQ ID NO: 1 and preferably at least the consecutive amino acid sequence Ile-Leu-Met may comprise more than two cysteines, e.g., four, six, eight, ten, twelve of more cysteines, preferably six cysteines, the cysteines are preferably present as disulfide bridges. It is however not excluded that there may be three, five, seven, nine, eleven, thirteen or more cysteines. The cysteines in the peptide according to the invention are preferably paired as the corresponding cysteines according to SEQ ID NO: 1, i.e. C3 is paired to C20; C10 is paired to C22 and C16 is paired to C28. The thermostable peptide according to the invention which inhibits contact activation of the blood clotting system may be any peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met may have any length. Preferably, the peptide according to the invention comprises at most 42, 41, 40, 39, 38, 37, 36, 35, 34, 33, 32, 31, 30, 29, 28, 27, 26, 25, 24, 23, 22, 21, 20 amino acids, more preferably between about 25-35 amino acids, more preferably about 27-32 amino acids, and most preferably about 29 amino acids.

When a peptide according to the invention is longer or shorter than the most preferred 29 amino acids, the net charge of the peptide according to the invention is preferably substantially identical to the net charge of SEQ ID NO: 1 when measured under identical conditions, preferably under physiological conditions. Substantially identical in this context means that the net charge of the peptides is preferably within +0.5 and −0.5, more preferably within +0.4 and −0.4, within +0.3 and −0.3, within +0.2 and −0.2, within +0.1 and −0.1 of the net charge of SEQ ID NO: 1 when measured under identical conditions, preferably under physiological conditions. Most preferably, the net charge of the peptide according to the invention is identical to the net charge of SEQ ID NO: 1 when measured under identical conditions, preferably under physiological conditions. Preferably, the pI of the peptide according to the invention is substantially identical to the pI of SEQ ID NO: 1. Substantially identical pI means that the pI of the peptide according to the invention is preferably within +0.5 and −0.5, more preferably within +0.4 and −0.4, within +0.3 and −0.3, within +0.2 and −0.2, within +0.1 and −0.1 of the pI of SEQ ID NO: 1.

The inhibition of contact activation is best measured by the prolongation of the activated partial thromboplastin time (aPTT). The aPTT is known to persons skilled in the art as a clotting time of plasma that is started by recalcification of citrated plasma in the presence of a substance that activates the contact system, such as kaolin or ellagic acid. It is described in detail by L. Poller in “Laboratory techniques in thrombosis” Edited by J. Jespersen, R. M. Bertina and F. Haverkate, ISBN 0-7923-5317-X (1999) p. 37-44. Alternatively the prolongation of the lag time of a thrombin generation experiment according to WO2003093831 can be determined. One functional unit of a contact activator inhibitor is defined as the amount that doubles the aPTT of the lag-time of a thrombin generation curve.

The minimal desired degree of inhibition of contact activation is the one that triples the lag time of a TG curve or an a PTT measurement.

Preferably, thermostability with respect to the thermostable peptide according to the invention is such that no activity of inhibition of contact activation of the blood clotting system is lost under conditions of heat-sterilisation, i.e. 120° C. for 20 minutes, as measured with a 0.1 mM solution of the thermostable peptide according to the invention in a closed vial. However, in general a 50% loss of activity can be accepted as long as the minimal degree of inhibition is still attained after sterilisation. In all the embodiments of the present invention, the thermostable peptide according to the invention which inhibits contact activation of the blood clotting system comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met, preferably retains at least 10, more preferably 20, 30, 40, 50, 60, 80, 90, 95 and most preferably 99% inhibition activity after heat-sterilization under conditions as described here above.

In all the embodiments of the present invention, the thermostable peptide according to the invention which inhibits contact activation of the blood clotting system may be present as a single peptide but different peptides according to the invention may be present; i.e. in all embodiments according to the invention it is envisioned that the peptides according to the invention may be mixed and applied in a mixture.

It will be evident that the composition according to the invention can conveniently be used for the collection of blood, to inhibit activation of the Ca²⁺, non physiological part of the clotting reaction sequence, i.e. the contact system. Accordingly, in a second aspect the present invention provides a device for collecting a sample comprising at least one calcium chelating substance with blood clotting inhibition activity and further comprising a thermostable peptide which inhibits contact activation of the blood clotting system, said peptide comprising within a consecutive sequence of at most about 40 amino acids at least two cysteines, the amino acid Arg or an analog of Arg on a position corresponding to amino acid position 5 in SEQ ID NO: 1 and preferably at least the consecutive amino acid sequence Ile-Leu-Met. Said device may be any device suitable for the collection or storage of a sample. Preferably, the sample is blood or blood plasma. If the sample is blood, the device is preferably a container for collection blood such as a tube, which may be a under vacuum in order to facilitate drawing blood from a subject. The device may also be a bag for collection of blood. Tubes (such as Becton Dickinson vacutainer glass, Sarstedt tubes; Terumo Venosafe, Becton Dickinson vacutainer plastic, Haemtech SCAT, Greiner vacuum tube) and bags for the collection of blood are known to the person skilled in the art.

Preferably, in the device according to the invention, the calcium-chelating substance with blood clotting inhibition activity and the thermostable peptide according to the invention which inhibits contact activation of the blood clotting system are present in concentrations and ratio's as described in the first aspect of the invention.

The composition and device according to the invention can conveniently be used to prevent contact activated clotting of blood when determining blood clotting activity in blood. Contact activation would bias the determination and investigation, especially while determination blood clotting activity by measuring thrombin activity over time (thrombin generation). Accordingly, in a third aspect the present invention provides a kit of parts comprising:

a device for collecting a sample comprising at least one calcium-chelating substance with blood clotting inhibition activity and further comprising a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met, and

a reagent for determining blood clotting activity, preferably the development of thrombin activity as a function of time.

Preferably, in the kit according to the invention, the device for collecting a sample may be any device, but is preferably a container for collection blood such as a tube, which may be a under vacuum in order to facilitate drawing blood from a subject.

In the kit according to the invention, the calcium chelating substance with blood clotting inhibition activity and the thermostable peptide according to the invention which inhibits contact activation of the blood clotting system are preferably present in concentrations and ratio's as described in the first aspect of the invention.

The kit may comprise any other reagent or device useful for the determination of blood clotting activity. The reagent for determining blood clotting activity may be any reagent known for that purpose to the person skilled in the art. When thrombin activity is measured, the reagent preferably is a fluorogenic thrombin substrate, preferably an oligopeptide coupled to a fluorogenic group. The general formula of such peptides is E-(AA)n-(Arg or Lys)-Fluo, where E is an end group, AA an amino acid and Fluo preferably aminomethylcoumarine or Rhodamine-110.

As described earlier herein, the thermostable peptide according to the invention can conveniently be used to inhibit contact activation of the blood clotting system. Accordingly, in a fourth aspect the present invention provides the use of a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met, for inhibiting activation of the blood clotting system through contact with foreign surfaces.

In the use according to the invention, the thermostable peptide according to the invention which inhibits contact activation of the blood clotting system may be contacted with the blood or any other biological fluid or a blood product such as or plasma as described earlier herein, in every way available to the person skilled in the art. The blood is preferably ex vivo blood or plasma, i.e. the taking of the blood has already taken place; the blood or plasma may have been stored. In the use according to the invention, the thermostable peptide according to the invention which inhibits contact activation of the blood clotting system is preferably present upon first contact of the blood with the foreign surface or as soon as possible after contacting with the blood, in concentrations and ratio's as described in the first aspect of the invention. Preferably, the thermostable peptide according to the invention is present together with a calcium chelating substance with blood clotting inhibition activity; the calcium-chelating substance with blood clotting inhibition activity and the thermostable peptide according to the invention which inhibits contact activation of the blood clotting system are preferably present in concentrations and ratio's as described in the first aspect of the invention. Preferably, the thermostable peptide according to the invention and the calcium-chelating substance with blood clotting inhibition activity are mixed with blood immediately after taking of blood in order to inhibit contact activation of the blood clotting system. This can conveniently be achieved by placing a thermostable peptide according to the invention and the calcium chelating substance with blood clotting inhibition activity in the container wherein the blood is collected. As such, the blood can be mixed immediately after the blood sample has been drawn. The container is preferably a device as described in the second aspect of the invention.

The thermostable peptide according to the invention can conveniently be used during the taking of a blood sample to inhibit contact activation of the blood clotting system, which clotting may otherwise already start during taking of the sample. Accordingly, in a fifth aspect the present invention provides the medical use of a thermostable peptide according to the invention. Namely, a thermostable peptide which inhibits contact activation of the blood clotting system, said peptide comprising within a consecutive sequence of at most about 40 amino acids at least two cysteines, the amino acid Arg or an analog of Arg on a position corresponding to amino acid position 5 in SEQ ID NO: 1 and preferably at least the consecutive amino acid sequence Ile-Leu-Met, for use as a medicament.

This aspect of the invention further provides a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met, for determining blood clotting activity, said method comprising:

collecting a blood sample form an individual, wherein said peptide is contacted with the blood sample,

measuring in said sample blood clotting activity, preferably thrombin activity, more preferably using a fluorescent assay for measuring thrombin activity, and preferably,

using the blood clotting activity value for diagnosis.

The sample may be any biological fluid as described earlier herein, preferably blood or plasma. The sample may be treated before measurement. As such, the sample may actually be a part of the original sample. Said part may be that only a portion of the sample is used and the rest of the sample is stored for future used. Said part may also mean that e.g. the measurement is performed on a supernatant of the sample, such as on plasma. The sample may also be stored between collection and measurement. As such, the measurement may e.g. be performed on plasma that has been stored at −80° C.

The blood clotting activity may be measured by any assay available to the person skilled in the art. Preferably, blood clotting activity is determined by measuring thrombin activity over time (thrombin generation). Thrombin activity may be measured by any means known to the person skilled in the art. A preferred assay uses a fluorogenic thrombin substrate, preferably an oligopeptide coupled to a fluorogenic group. The general formula of such peptides is E-(AA)n-(Arg or Lys)-Fluo, where E is an end group, AA an amino acid and Fluo preferably aminomethylcoumarine or Rhodamine-110 for measuring thrombin activity. Such assay is described in WO2003/093831 for plasma and in WO2006/117246 for whole blood. In a typical assay two parts of plasma or blood are mixed with one part of a solution containing at least Ca²⁺, a fluorogenic substrate as described above and tissue factor so as to start thrombin generation.

The value of the blood clotting activity preferably is used for the diagnosis of several procoagulant and anticoagulant conditions, such as congenital or acquired thrombophilia, congenital or acquired hemophilia, consumption coagulopathy, or the effect of antithrombotic drugs (such as heparin, vitamin K antagonists, direct thrombin inhibitors) or procoagulant drugs (such as concentrates of factor VIII or IX). The normal value of the endogenous thrombin potential, i.e. the area under the thrombin generation curve, when tested by the method known to the art as examplified in example 1, 1600±250 nM·min. Values below 400 nM·min indicate a definite bleeding tendency, values above 1850 nM·min a thrombotic tendency.

In the medical use according to the invention, the thermostable peptide according to the invention which inhibits contact activation of the blood clotting system is contacted with the blood or other biological fluid as described earlier herein. The thermostable peptide according to the invention may be contacted with the blood or other biological fluid as described earlier herein in every way available to the person skilled in the art. After contacting with the blood, plasma or other biological fluid as described earlier herein, the thermostable peptide according to the invention which inhibits contact activation of the blood clotting system is preferably present in concentrations and ratio's as described in the first aspect of the invention. Preferably, the thermostable peptide according to the invention is present together with a calcium-chelating substance with blood clotting inhibition activity; the calcium-chelating substance with blood clotting inhibition activity and the thermostable peptide according to the invention which inhibits contact activation of the blood clotting system are preferably present in concentrations and ratio's as described in the first aspect of the invention. Preferably, the thermostable peptide according to the invention and (if used) the calcium-chelating substance with blood clotting inhibition activity are mixed with blood during taking of blood in order to inhibit contact activation of the blood clotting system. This can conveniently be achieved by placing a thermostable peptide according to the invention and a calcium chelating substance with blood clotting inhibition activity in the container wherein the blood is collected. As such, the blood can be mixed immediately after the blood sample has been drawn. The container is preferably a device as described in the second aspect of the invention.

As described earlier herein, the thermostable peptide according to the invention can conveniently be used to inhibit contact activation of the blood clotting system. Accordingly, in a sixth aspect the present invention provides a method for inhibiting clotting of blood, the method comprising contacting a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met, with blood, plasma or any other biological fluid described earlier herein, in an amount sufficient to inhibit the clotting.

In the method for inhibiting clotting of blood according to the invention, the thermostable peptide according to the invention which inhibits contact activation of the blood clotting system is contacted with the blood or other biological fluid as described earlier herein The thermostable peptide according to the invention may be contacted with the blood in every way available to the person skilled in the art. After contacting with the blood, the peptide according to the invention which inhibits contact activation of the blood clotting system is preferably present in an amount sufficient to inhibit clotting; preferably the thermostable peptide according to the invention is present in concentrations and ratio's as described in the first aspect of the invention. Preferably, the thermostable peptide according to the invention is present together with a calcium chelating substance with blood clotting inhibition activity; the calcium chelating substance with blood clotting inhibition activity and the thermostable peptide according to the invention which inhibits contact activation of the blood clotting system are preferably present in concentrations and ratio's as described in the first aspect of the invention. The thermostable peptide according to the invention may be contacted with the blood or other biological fluid as described earlier herein at any time. This may be performed immediately when taking the sample, but may also be performed later including after storage of the sample, e.g. before measurement of a parameter such as clotting activity.

As described earlier herein, a thermostable peptide according to the invention can conveniently be used to inhibit contact activation of the blood clotting system. Therefore, said peptide can conveniently be used to collect and prepare a sample while inhibiting contact activation of the blood clotting system in said sample.

Accordingly, in a seventh aspect the present invention provides a method for collection and preparation of a sample from an individual comprising:

providing a device for collecting a sample, said device comprising at least one calcium chelating substance with blood clotting inhibition activity and further comprising a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met,

collecting a sample into the device, and

mixing sample, calcium binding substance with blood clotting inhibition activity and peptide.

The device may be any device known to the person skilled in the art and suitable for the purpose of collection of a sample. Preferably, the device is a device as described in the second aspect of the invention. The sample may be any biological sample such as a biological fluid described earlier herein; preferably the sample is blood or plasma. In the device, the calcium chelating substance with blood clotting inhibition activity and the peptide according to the invention which inhibits contact activation of the blood clotting system are preferably present in concentrations and ratio's as described in the first aspect of the invention.

The sample, preferably blood, may be collected by any means known to the person skilled in the art, e.g. blood may be collected from an individual through a hollow needle which may be connected to the device.

After collection, or even during collection, the sample, preferably blood is mixed with the calcium-chelating substance with blood clotting inhibition activity and the thermostable peptide according to the invention. Mixing may be performed by any means known to the person skilled in the art. The device may be tilted by hand one or more timed. The device may also be placed in a mechanism or tool for mixing purposes.

As described earlier herein, a thermostable peptide according to the invention inhibits contact activation of blood, while not inhibiting thromboplastin induced thrombin generation, i.e. not impeding the normal clotting process. Therefore, a thermostable peptide according to the invention can conveniently be used in an assay wherein blood clotting activity is determined. Accordingly, in an eighth aspect, the present invention provides a method for determining blood clotting activity in a blood or plasma sample, or any other biological fluid as described earlier herein, the method comprising: measuring in said sample comprising at least one calcium chelating substance with blood clotting inhibition activity and further comprising a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met, blood clotting activity, preferably thrombin activity, more preferably using a fluorescent assay for measuring thrombin activity. In the blood sample to be analyzed for clotting activity, the thermostable peptide according to the invention may already be present or may be added at a suitable time before analysis. In an embodiment, the blood sample has been collected in a device according to the second aspect of the invention. In such device the thermostable peptide according to the invention is already present when the blood sample is taken. As such, the blood is immediately mixed with the thermostable peptide according to the invention; thus contact activation of the blood clotting system is inhibited. Preferably, the blood or plasma sample, or any other biological fluid as described earlier herein, comprises both a thermostable peptide according to the invention and a calcium-chelating substance with blood clotting inhibition activity; the calcium-chelating substance with blood clotting inhibition activity and the thermostable peptide according to the invention which inhibits contact activation of the blood clotting system are preferably present in concentrations and ratio's as described in the first aspect of the invention.

The blood clotting activity may be measured by any assay available to the person skilled in the art. Preferably, blood clotting activity is determined by measuring thrombin activity over time (thrombin generation). Thrombin activity may be measured by any means known to the person skilled in the art. A preferred assay uses a fluorogenic thrombin substrate, preferably aminomethylcoumarine for measuring thrombin activity. Such assay is described in WO2003/093831 for plasma and in WO2006/117246 for blood. See e.g. Example 1 herein.

In a ninth aspect, the present invention provides a thermostable peptide which inhibits contact activation of the blood clotting system, said peptide comprising within a consecutive sequence of at most about 40 amino acids at least two cysteines, the amino acid Arg or an analog of Arg on a position corresponding to amino acid position 5 in SEQ ID NO: 1 and preferably at least the consecutive amino acid sequence Ile-Leu-Met.

According to the invention, in a composition according to the first aspect of the invention, a device according to the second aspect of the invention, a kit according to the third aspect of the invention, a use according to the fourth aspect of the invention, a peptide for medical use according to the fifth aspect of the invention, a method according to any one of the sixth, seventh and eighth aspect of the invention or a peptide according to the ninth aspect of the invention, the thermostable peptide which inhibits contact activation of the blood clotting system, preferably comprises within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met.

According to the invention, in a composition according to the first aspect of the invention, a device according to the second aspect of the invention, a kit according to the third aspect of the invention, a use according to the fourth aspect of the invention, a peptide for medical use according to the fifth aspect of the invention, a method according to any one of the sixth, seventh and eighth aspect of the invention or a peptide according to the ninth aspect of the invention, the thermostable peptide which inhibits contact activation of the blood clotting system, preferably comprises a consensus amino acid sequence: XXCXXXXXXCXXXXXCXXXCXCXXXXXCX (SEQ ID NO: 9), wherein X at position 5 is R or an analog of R; X at position 9 is E or K, preferably K and X at position 21 is I or V, preferably V; or more preferably: XILMXCKKDSDCLAECX (SEQ ID NO: 6), wherein X at position 1 is K or R or an analog of R, preferably R or an analog of R; X at position 5 is E or K, preferably K and X at position 17 is I or V, preferably V; or more preferably: RVCPXILMXCKKDSDCLAECXCLEHGYCG (SEQ ID NO: 7), wherein X at position 5 is K or R or an analog of R, preferably R or an analog of R; X at position 9 is E or K, preferably K and X at position 21 is I or V, preferably V. In the embodiment above defining SEQ ID NO:9, it is further preferred that SEQ ID NO:9 has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:1.

In an embodiment, the consensus amino acid sequence SEQ ID NO:9 comprises or consists of or is a consensus amino acid sequence:

XXCXXXXXXCXXXXXCXXXCXCXXXXXCX,

wherein X at position 5 is R or an analog of R,
wherein X at position 1 is R or an analog or R,
wherein X at position 2 is V or an analog of V,
wherein X at position 9 is E or K, preferably K,
wherein X at position 21 is I or V, preferably V, and/or
wherein X at position 4 is P or an analog of P and preferably
   said SEQ ID NO:9 has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:1.

In this embodiment, the consensus amino acid sequence may comprise or consist of:

(SEQ ID NO: 10)
RXCXXXXXXCXXXXXCXXXCXCXXXXXCX
(SEQ ID NO: 11)
XVCXXXXXXCXXXXXCXXXCXCXXXXXCX
(SEQ ID NO: 12)
XXCPXXXXXCXXXXXCXXXCXCXXXXXCX
(SEQ ID NO: 13)
XXCXRXXXXCXXXXXCXXXCXCXXXXXCX
(SEQ ID NO: 14)
RVCXXXXXXCXXXXXCXXXCXCXXXXXCX
(SEQ ID NO: 15)
RXCPXXXXXCXXXXXCXXXCXCXXXXXCX
(SEQ ID NO: 16)
RXCXRXXXXCXXXXXCXXXCXCXXXXXCX
(SEQ ID NO: 17)
XVCPXXXXXCXXXXXCXXXCXCXXXXXCX
(SEQ ID NO: 18)
XVCXRXXXXCXXXXXCXXXCXCXXXXXCX
(SEQ ID NO: 19)
XXCPRXXXXCXXXXXCXXXCXCXXXXXCX
(SEQ ID NO: 20)
RXCXRXXXXCXXXXXCXXXCXCXXXXXCX
(SEQ ID NO: 21)
RVCPXXXXXCXXXXXCXXXCXCXXXXXCX
(SEQ ID NO: 22)
RVCXRXXXXCXXXXXCXXXCXCXXXXXCX
(SEQ ID NO: 23)
XVCPRXXXXCXXXXXCXXXCXCXXXXXCX
(SEQ ID NO: 24)
RXCPRXXXXCXXXXXCXXXCXCXXXXXCX
(SEQ ID NO: 25)
RVCPRXXXXCXXXXXCXXXCXCXXXXXCX,,

wherein in each of SEQ ID NO: 10-25,
X at position 9 is E or K, preferably K and X at position 21 is I or V, preferably V, and preferably wherein each of SEQ ID NO: 10-25 has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:1. In this paragraph, it is to be understood that R, V or P as identified in SEQ ID NO:10-25 have the same meaning as in SEQ ID NO:9: R may be R or an analog of R, V may be V or an analog of V and P may be P or an analog of P.

The person skilled in the art will comprehend that conservative substitutions of amino acids can be made without changing the functional features of said peptide. Such conservative substitutions are within the scope of the present invention as long as the peptide remains a thermostable peptide which inhibits contact activation of the blood clotting system. Thermostability is preferably as defined earlier herein.

According to the invention, in a composition according to the first aspect of the invention, a device according to the second aspect of the invention, a kit according to the third aspect of the invention, a use according to the fourth aspect of the invention, a peptide for medical use according to the fifth aspect of the invention, a method according to any one of the sixth, seventh and eighth aspect of the invention or a peptide according to the ninth aspect of the invention, the thermostable peptide which inhibits contact activation of the blood clotting system, preferably comprises a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with a peptide as shown in SEQ ID NO: 1, SEQ ID NO: 2, SEQ ID NO: 3, or SEQ ID NO: 4.

More preferably, the peptide comprises a peptide with the sequence as shown in SEQ ID NO: 1 or SEQ ID NO: 2. Even more preferably, the peptide comprises a peptide with the sequence as shown in SEQ ID NO:1.

According to the invention, in a composition according to the first aspect of the invention, a device according to the second aspect of the invention, a kit according to the third aspect of the invention, a use according to the fourth aspect of the invention, a peptide for medical use according to the fifth aspect of the invention, a method according to any one of the sixth, seventh and eighth aspect of the invention or a peptide according to the ninth aspect of the invention, the thermostable peptide which inhibits contact activation of the blood clotting system, preferably is a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with a peptide as shown in SEQ ID NO: 1, SEQ ID NO: 2, SEQ ID NO: 3, or SEQ ID NO: 4.

More preferably, the peptide is a peptide with the sequence as shown in SEQ ID NO: 1 or SEQ ID NO: 2. Even more preferably, the peptide is a peptide with the sequence as shown in SEQ ID NO:1.

The invention further provides a polynucleotide comprising a polynucleotide encoding a thermostable peptide according to the invention.

Preferably, the polynucleotide comprises a polynucleotide encoding a thermostable peptide which inhibits contact activation of the blood clotting system, said peptide comprising within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met.

More preferably, the polynucleotide comprises a polynucleotide encoding a thermostable peptide which comprises within a consecutive sequence of at most about 40 amino acids at least two or at least four cysteines, the amino acid Arg or an analog of Arg on a position corresponding to amino acid position 5 in SEQ ID NO: 1 and preferably at least the consecutive amino acid sequence Ile-Leu-Met.

Even more preferably, the polynucleotide comprises a polynucleotide encoding a peptide which comprises a consensus amino acid sequence: XXCXXXXXXCXXXXXCXXXCXCXXXXXCX (SEQ ID NO: 9), wherein X at position 5 is R or an analog of R, preferably R or an analog of R; X at position 9 is E or K, preferably K and X at position 21 is I or V, preferably V; or more preferably: XILMXCKKDSDCLAECX (SEQ ID NO: 6), wherein X at position 1 is K or R, preferably R; X at position 5 is E or K, preferably K and X at position 17 is I or V, preferably V; or more preferably: RVCPXILMXCKKDSDCLAECXCLEHGYCG (SEQ ID NO: 7), wherein X at position 5 is K or R, preferably R; X at position 9 is E or K, preferably K and X at position 21 is I or V, preferably V. In the embodiment above defining a polynucleotide of the invention, said polynucleotide encoding SEQ ID NO:9, it is further preferred that a polynucleotide of the invention encodes SEQ ID NO:9 or encodes a peptide that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:1.

In an embodiment, the polynucleotide encodes a consensus amino acid sequence SEQ ID NO:9, said SEQ ID NO:9 comprising or consisting of or being a consensus amino acid sequence: XXCXXXXXXCXXXXXCXXXCXCXXXXXCX,
wherein X at position 5 is R or an analog of R,
wherein X at position 1 is R or an analog or R,
wherein X at position 2 is V or an analog of V,
wherein X at position 9 is E or K, preferably K,
wherein X at position 21 is I or V, preferably V, and/or
wherein X at position 4 is P or an analog of V and preferably
   said SEQ ID NO:9 has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:1.

In an embodiment, it is preferred that said polynucleotide encodes a consensus amino acid sequence comprising or consisting of:

(SEQ ID NO: 10)
RXCXXXXXXCXXXXXCXXXCXCXXXXXCX
(SEQ ID NO: 11)
XVCXXXXXXCXXXXXCXXXCXCXXXXXCX
(SEQ ID NO: 12)
XXCPXXXXXCXXXXXCXXXCXCXXXXXCX
(SEQ ID NO: 13)
XXCXRXXXXCXXXXXCXXXCXCXXXXXCX
(SEQ ID NO: 14)
RVCXXXXXXCXXXXXCXXXCXCXXXXXCX
(SEQ ID NO: 15)
RXCPXXXXXCXXXXXCXXXCXCXXXXXCX
(SEQ ID NO: 16)
RXCXRXXXXCXXXXXCXXXCXCXXXXXCX
(SEQ ID NO: 17)
XVCPXXXXXCXXXXXCXXXCXCXXXXXCX
(SEQ ID NO: 18)
XVCXRXXXXCXXXXXCXXXCXCXXXXXCX
(SEQ ID NO: 19)
XXCPRXXXXCXXXXXCXXXCXCXXXXXCX
(SEQ ID NO: 20)
RXCXRXXXXCXXXXXCXXXCXCXXXXXCX
(SEQ ID NO: 21)
RVCPXXXXXCXXXXXCXXXCXCXXXXXCX
(SEQ ID NO: 22)
RVCXRXXXXCXXXXXCXXXCXCXXXXXCX
(SEQ ID NO: 23)
XVCPRXXXXCXXXXXCXXXCXCXXXXXCX
(SEQ ID NO: 24)
RXCPRXXXXCXXXXXCXXXCXCXXXXXCX
(SEQ ID NO: 25)
RVCPRXXXXCXXXXXCXXXCXCXXXXXCX,,

wherein in each of SEQ ID NO: 10-25,
X at position 9 is E or K, preferably K and X at position 21 is I or V, preferably V, and preferably wherein each of SEQ ID NO: 10-25 has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:1. In this paragraph, it is to be understood that R, V or P as identified in SEQ ID NO:10-25 have the same meaning as in SEQ ID NO:9: R may be R or an analog of R, V may be V or an analog of V and P may be P or an analog of P.

The person skilled in the art will comprehend that conservative substitutions of amino acids can be made without changing the functional features of said peptide. Such conservative substitutions are within the scope of the present invention as long as the peptide remains a thermostable peptide which inhibits contact activation of the blood clotting system. Thermostability is preferably as defined earlier herein.

Even more preferably, the polynucleotide comprises a polynucleotide encoding a thermostable peptide which comprises a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with a peptide as shown in SEQ ID NO: 1, SEQ ID NO: 2, SEQ ID NO: 3, or SEQ ID NO: 4. More preferably, the peptide comprises a peptide with the sequence as shown in SEQ ID NO: 1 or SEQ ID NO: 2. Even more preferably, the peptide comprises a peptide with the sequence as shown in SEQ ID NO:1.

Even more preferably, the polynucleotide comprises a polynucleotide encoding a thermostable peptide which is a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with a peptide as shown in SEQ ID NO: 1, SEQ ID NO: 2, SEQ ID NO: 3, or SEQ ID NO: 4. More preferably, the peptide is a peptide with the sequence as shown in SEQ ID NO: 1 or SEQ ID NO: 2. Even more preferably, the peptide is a peptide with the sequence as shown in SEQ ID NO:1.

The polynucleotide according to the invention may be any type of polynucleotide, including single-stranded, double stranded DNA, RNA or the like. The polynucleotide may be comprised in a nucleic acid construct or in a vector which may comprise additional polynucleotides including control sequences that can induce expression of the polynucleotide according to the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 0.1% of the value.

The sequence information as provided herein should not be so narrowly construed as to require inclusion of erroneously identified amino acids. The skilled person is capable of identifying such erroneously identified amino acids and knows how to correct for such errors.

All patent and literature references cited in the present specification are hereby incorporated by reference in their entirety.

### FIGURE LEGENDS

FIG. 1: Effect of the natural TICA-1 on thrombin generation triggered by tissue factor and by contact activation. Drawn lines: No TICA1; dotted lines: 7.5 μg/ml TICA1. From left to right: Tissue factor triggered thrombin generation (2 overlapping peaks); contact activated thrombin generation (no TICA1); contact activated thrombin (with TICA1).

FIG. 2: Contact activated thrombin generation, inhibition by CTI and TICA1 Drawn lines depict TICA-1. Concentrations from left to right: none, 1, 2, 3 and 5 μM. Dotted line depicts CTI 0.75 μM.

FIG. 3: Quantification of the inhibitory effects on factor XIIa. Circles represent data points; line represents best fit.

3a: effect of TICA1 (PT1) on hydrolysis of S2302 by h-FXIIa; FXIIa=5 nM; Kcal=25 s⁻¹; Km=180 μM; Ki=251 nM

3b: effect of CTI on hydrolysis of S2302 by h-FXIIa; FXIIa=4.21 nM; Kcat=25 s⁻¹; Km=180 μM; Ki=2.90 nM

FIG. 4: Oxidative folding of TICA-1.

4a: HPLC of reduced (thick line) and oxidized (thin line) TICA-1 showing a typical reduction of retention time as a result of protein folding.

4b: Folded TICA-1; 4c: Unfolded TICA-1. Folding of TICA-1 results in a mass decrease of ˜6 Da, corresponding to the loss of 6 protons due to the formation of 3 disulfide bonds.

FIG. 5: Thermostable Inhibitor of Coagulation Activation (TICA1): Dependence on Disulfides.

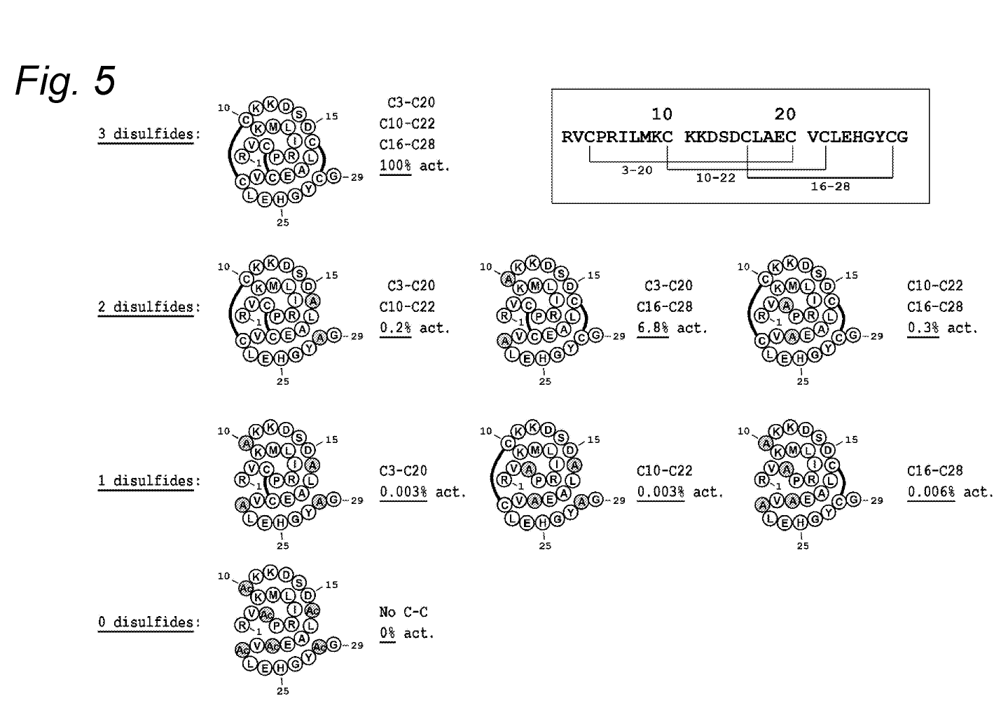

3 disulfides (C3-C20, C10-C22, C16-C28): 100% activity

2 disulfides from 0.2 to 6.8% activity:

(C3-C20, C10-C22): 0.2% activity

(C10-C22, C16-C28): 0.3% activity

(C3-C20, C16-C28): 6.8% activity

1 disulfide from 0.003 to 0.006% activity

(C10-C22): 0.003% activity

(C3-C20): 0.003% activity

(C16-C28): 0.006% activity

No disulfide, no C: 0 activity

FIG. 6: TICA1: Ala-scan with intact disulfide bonds.

### SEQUENCES

### Sequences as Set Forth in the Sequence Listing

| SEQ | | |
|---|---|---|
| ID | | |
| NO: | ID | Gene product |
| 1 | TICA 1 | RVCPRILMKCKKDSDCLAECVCLEHGYCG |
| 2 | TICA 2 | RVCPRILMECKKDSDCLAECVCLEHGYCG |
| 3 | TICA 3 | HEERVCPRILMKCKKDSDCLAECVCLEHG |
| | | YCG |
| 4 | TICA 4 | RVCPKILMECKKDSDCLAECICLEHGYCG |
| 5 | TICA 5 | HEERVCPKILMECKKDSDCLAECICLEHG |
| | | YCG |
| 6 | Consensus | XILMXCKKDSDCLAECX |
| | short | |
| 7 | Consensus | RVCPXILMXCKKDSDCLAECXCLEHGYCG |
| | long | |
| 8 | PCTI | RVCGIGPRPRLPWPRILMKCKKDSDCLAE |
| | | CVCLEHGYCG |
| 9 | Consensus | XXCXXXXXXCXXXXXCXXXCXCXXXXXCX |

Wherein in SEQ ID NO: 6, X at position 1 is K or R, preferably R; X at position 5 is E or K, preferably K and X at position 17 is I or V, preferably V.
Wherein in SEQ ID NO: 7, X at position 5 is K or R, preferably R; X at position 9 is E or K, preferably K and X at position 21 is I or V, preferably V.
Wherein in SEQ ID NO: 9, X at position 5 is R or an analog of R; X at position 9 is E or K, preferably K and X at position 21 is I or V, preferably V.

The present invention is further described by the following examples which should not be construed as limiting the scope of the invention.

Unless stated otherwise, the practice of the invention will employ standard conventional methods of molecular biology, virology, microbiology or biochemistry. Such techniques e.g. are described in Sambrook et al. (1989) Molecular Cloning, A Laboratory Manual (2^{nd} edition), Cold Spring Harbor Laboratory, Cold Spring Harbor Laboratory Press; in Sambrook and Russell (2001) Molecular Cloning: A Laboratory Manual, Third Edition, Cold Spring Harbor Laboratory Press, NY; in Volumes 1 and 2 of Ausubel et al. (1994) Current Protocols in Molecular Biology, Current Protocols, USA; and in Volumes I and II of Brown (1998) Molecular Biology LabFax, Second Edition, Academic Press (UK); “Laboratory techniques in thrombosis” Edited by J. Jespersen, R. M. Bertina and F. Haverkate, ISBN 0-7923-5317-X (1999); “Haemostasis and thrombosis: Basic principles and clinical practice” Eds: Colman, Hirsh, Marder, Clowes, George Lippincott Williams and Wilkins. (2010).

### EXAMPLES

### Summary

We isolated the trypsin inhibitors (serpins) from a multitude of seeds by affinity chromatography on a trypsin column and further purification procedures known to the art, notably the serpin from Cucurbita Maxima seeds, i.e. squash trypsin inhibitor (STI) (see example 2).

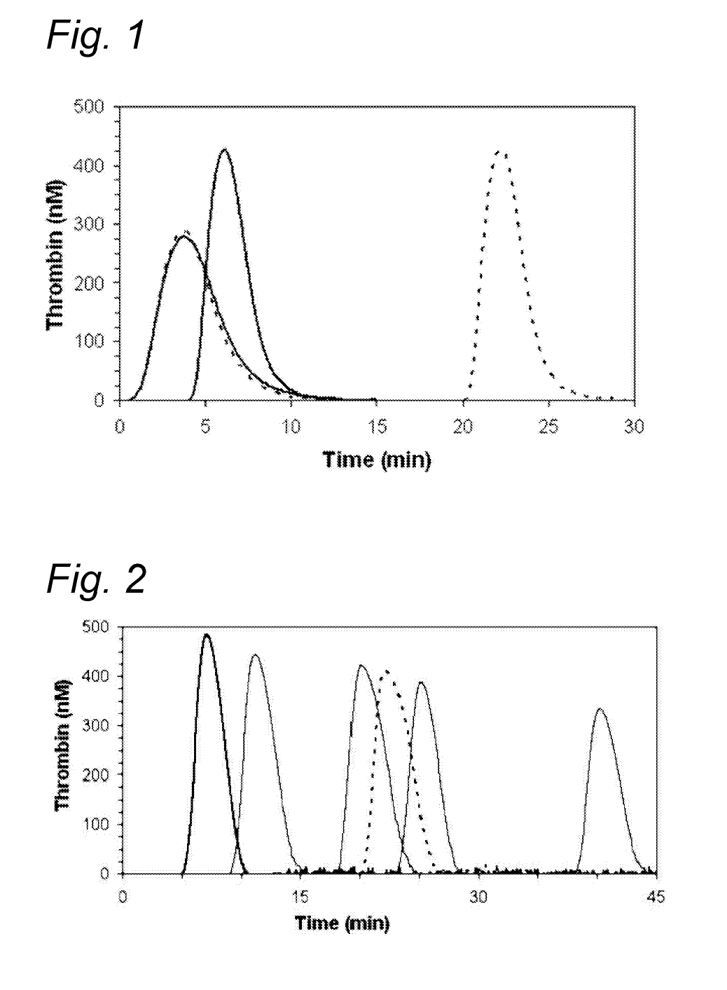

We confirmed that the serpin is an inhibitor of factor XIIa (see example 3). We observed that it effectively inhibited thrombin generation that was initiated by contact activation but not TG that was started by the natural trigger tissue factor (see FIG. 1). It should be noted here that inhibition of contact activation is seen in thrombin generation curves as a prolongation of the lag time of thrombin generation because contact activation produces factor IXa and a certain critical amount of factor IXa is required to start thrombin generation.

That tissue factor driven thrombin generation is not inhibited indicates that STI does not inhibit the natural clotting process but only contact activation. Indeed STI inhibited contact activation so well that blood taken on a solution not containing sodium citrate or another substance that binds Ca²⁺ ions but only STI (10 μg/ml) remained fluid for over 45 minutes, under conditions where in absence of STI it would clot in less than 5 min. If a tube containing STI was sterilized by heating for one hour in boiling water the STI, surprisingly, did not loose its inhibitory activity: blood would still clot after >45 min only. CTI under similar circumstances, at 10 μg/ml, lost much of its activity because a clotting time of <15 min. was observed.

The natural product was further analysed and found to contain two active polypeptides, which were purified to homogeneity (example 2).

These two natural peptides (TICA1 and TICA2) were further analysed to determine their primary structure by means of Tandem Matrix-assisted Laser Desorption Ionisation Time-of-flight (MALDI-TOF) mass spectrometry in conjunction with peptide mass fingerprinting (PMF) of fragments obtained by trypsinolysis. PMF positive fragments were confirmed by collision-induced fragmentation (CID) and amino acid sequence determination. The primary structures found is shown in table 1.

TABLE 1
Aminoacid sequence and Molecular
Weight (MW) of TICA 1-5

| SEQ | | | | |
|---|---|---|---|---|
| ID | | | MW | MW |
| NO: | ID | Gene product | Reduced | folded |
| 1 | TICA 1 | RVCPRILMKCKKDSDCLAECVC | 3274 | 3268 |
| | | LEHGYCG | | |
| 2 | TICA 2 | RVCPRILMECKKDSDCLAECVC | 3275 | 3269 |
| | | LEHGYCG | | |
| 3 | TICA 3 | HEERVCPRILMKCKKDSDCLAE | 3669 | 3663 |
| | | CVCLEHGYCG | | |
| 4 | TICA 4 | RVCPKILMECKKDSDCLAECIC | 3260 | 3254 |
| | | LEHGYCG | | |
| 5 | TICA 5 | HEERVCPKILMECKKDSDCLAE | 3656 | 3650 |
| | | CICLEHGYCG | | |

TICA 1 and 2 we then synthesized by means of Solid Phase Peptide Synthesis together with several congeners (Table 1, TICA 3, 4 and 5) (see example 4).

We determined the inhibitory action of the synthetic materials in the same way as we had done for the natural ones and found TICA1 to be the most active (see table 2 and example 3).

TABLE 2
Inhibitory activity of different polypeptides

| Inhib- | | | | | | | |
|---|---|---|---|---|---|---|---|
| itor | CTI | STI | TICA1 | TICA2 | TICA3 | TICA4 | TICA5 |
| Ki (nM) | 2.9 | 251 | 25.5 | 109 | 3090 | 1420 | 171000 |

We then determined the action of TICA1 as an inhibitor of contact activation. It appeared to prolong the lag time of thrombin generation that was triggered by contact activation, in the same manner as STI did (see FIG. 1).

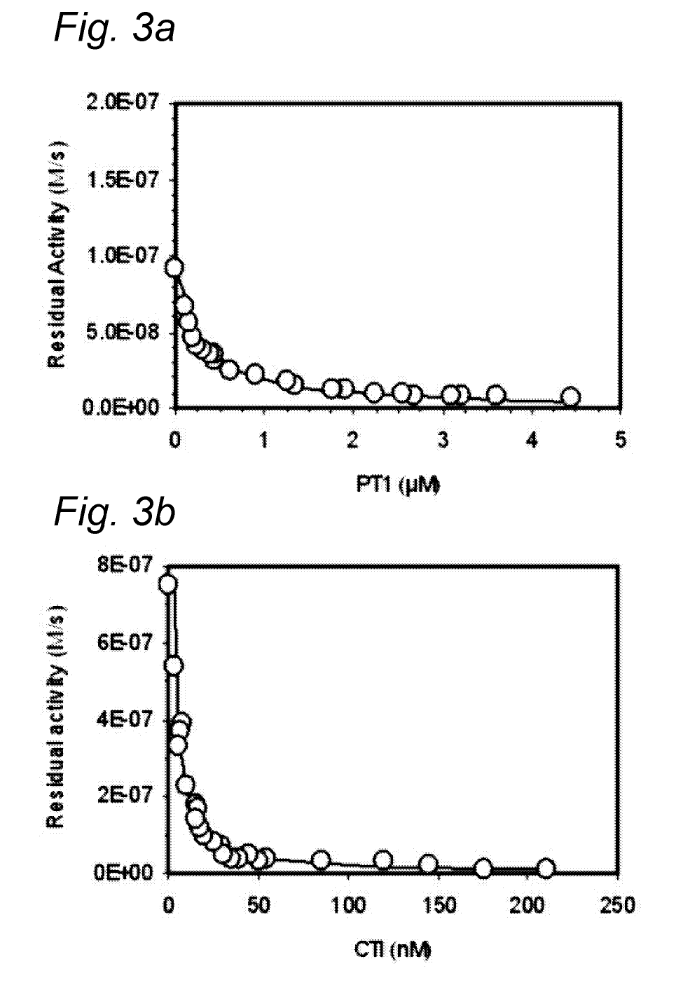

TICA1 was further modified by inserting the CTI sequence around the reactive bond, PCTI (SEQ ID NO: 8). The resulting PCTI peptide was inactive indicating that the new found inhibitors have no structure-function relationship in common with CTI. The effect of TICA1 on contact activation of PPP, elicited by incubation with kaolin, was compared with the effect of CTI. As can be seen in table 3 and FIG. 2, there is a dose-dependent inhibition of contact activation when PPP is incubated with various concentrations of TICA1 (0.94 to 4.6 μM). On a molar basis, peptide B is ˜2.5 times less potent than CTI is. On a weight basis it is equipotent.

TABLE 3
Influence of TICA1 on contact activation.

| Added | μM | μg/ml | Lag-time | ETP | Peak | ttPeak |
|---|---|---|---|---|---|---|
| TF | | | 1.5 | 1250 | 374 | 3 |
| Kaolin | | | 4.81 | 1209 | 454 | 6.06 |
| Kaolin-CTI | 0.72 | 8.64 | 18.62 | 1116 | 385 | 20.06 |
| Kaolin-PTI | 0.94 | 3.76 | 9.81 | 1146 | 419 | 11.06 |
| Kaolin-PTI | 1.87 | 7.48 | 17.69 | 1144 | 405 | 18.94 |
| Kaolin-PTI | 2.81 | 11.24 | 23.88 | 1092 | 375 | 25.38 |
| Kaolin-PTI | 3.74 | 14.96 | 19.69 | 1070 | 355 | 21.25 |
| Kaolin-PTI | 4.68 | 18.72 | 38.69 | 949 | 324 | 40.19 |

TICA1 has a MW=3268 Da and is easy to synthesize, fold and purify.

It can be left for weeks at room temperature without loosing activity. Heating a 0.1 mM solution in a closed ampoule in boiling water for 60 min does not affect its biological activity whereas CTI is significantly inactivated. (Example 7) This means that, unlike CTI, it can be added to citrate tubes that can be sterilized and used under clinical conditions. About 0.10 mg of TICA1 would be required per vacuum tube meant to take 10 ml of blood.

We thus provided a class of polypeptides, the TICAs (Thermostable Inhibitors of Contact Activation) that are specific inhibitors of the contact activation system of blood coagulation and therefore can be profitably used to prevent this system from disturbing the function analysis of thrombin generation in blood samples in the same manner as the previously patented corn trypsin inhibitor, with the additional advantage of heat stability and hence the possibility of sterilisation which greatly enhances its use in routine medical diagnostic practice.

### Example 1

Thrombin generation in presence of the various polypeptides.

Thrombin generation was carried out according to WO2003/093831.

Venous blood was collected into tubes containing 0.106 mol/l tri-sodium citrate (1:9, v:v). from healthy adult volunteers after obtaining informed consent. Following a double centrifugation at 2500×g for 15 min at room temperature, platelet poor plasma (PPP) was collected from the upper half volume of plasma supernatant, quick frozen and stored at −80° C. The absence of platelet and leucocyte in PPP was checked with an ADVIA 120 counter (Bayer Diagnostics, NY, USA).

Reagents for Thrombin Generation Test

Recombinant human tissue factor Innovin® was obtained from Dade Behring (Marburg, Germany) and used at a final concentration of 0.5 pM in PRP and 1 or 5 pM in PPP samples. The phospholipid vesicles used at a final concentration of 4 were obtained from Avanti Polar Lipids (Alabaster, Ala., USA) and consisted of 20 mol % phosphatidylserine (PS), 20 mol % phosphatidylethanolamine (PE) and 60 mol % phosphatidylcholine (PC) and were prepared by extrusion method (9,10). Kaolin light (hydrated aluminium silicate) was obtained from Baker Harrison ltd, (Ilford, UK).

Hepes-buffered saline contained 20 mM Hepes (Sigma Aldrich, l'Ile d'Abeau Chesnes, France), 140 mM NaCl and 5 mg/mL bovine serum albumin (BSA) (Euromedex, Souffelweyersheim, France), pH 7.35. This buffer was stored at −20° C. until use. A fresh mixture of fluorogenic substrate and CaCl₂ was prepared before each experiment. Fluorogenic substrate, Z-Gly-Gly-Arg-AMC, was obtained from Bachem (Bubendorf, Switzerland). The mixture of fluorogenic substrate 2.5 mM and CaCl₂ 0.1 M was prepared using buffer containing Hepes 20 mM and 60 mg/mL BSA, pH 7.35. The Calibrator with the activity of 600 nM human thrombin was obtained from Thrombinoscope BV (Maastricht, The Netherlands). Transparent, round bottom Greiner microtiter plates (Greiner ref 65204, Poitiers, France) were used.

Calibrated Automated Measurement of Thrombin Generation (CAT)

Thrombin generation was measured using Calibrated Automated Thrombography (CAT) according to the methodology of WO2003/093831 and a 96-well plate fluorometer (Fluoroscan Ascent Reader, Thermolab systems OY, Helsinki, Finland) equipped with a 390/460 nm filter set. Briefly, 80 μL of plasma is dispensed into the wells of round-bottom 96 well-microtiter plates. 20 μL of a mixture containing tissue factor and phospholipids is added to the PPP sample; alternatively, in order to obtain contact activation, the citrated plasma is preincubated for at least 10 min with 1 mg/ml of Kaolin light. The starting reagent (20 μL per well) contains fluorogenic substrate and CaCl₂. A dedicated software program, Thrombinoscope® (Thrombinoscope by, Maastricht, The Netherlands) enabled the calculation of thrombin activity against the calibrator (Thrombinoscope by, Maastricht, The Netherlands) and displayed thrombin activity with the time. The most important parameters that can be derived from CAT are lag time, endogenous thrombin potential corresponding to the area under the CAT curve, peak height of thrombin and time to peak.

### Example 2

### Purification of the Serpin STI from Curbita Maxima Seeds

STI is purified from Cucurbita maxima seeds in a three step procedure:

1: Extraction of crushed seeds with 0.1 M TRIS buffer, pH 8.0 and

2: Affinity chromatography on a Trypsin-Sepharose column

3: Reversed Phase (RP) HPLC—C18 column, linear gradient 0-60% acetonitrile in water 0.1% trifluoroacetic acid

This allows the separation of two active peptides (TICA1 and TICA2) of which the amino-acid sequence was determined (see Table 1).

### Example 3

### Inhibition of Factor XIIa by Serpins

Materials
S2302 From Chromogenic, lot # N0398718 SEQ0860, exp. date 2012-07. Water added on 7 Oct. 2010→3.7 mM. On 3 Aug. 10 the OD at 316 nm was measured (0.236+0.235)/2×200=47.1. The calculated concentration thus is 47.1/12.9=3.7 mM. The kinetics of S2302 hydrolysis by FXIIa was Kₘ=180 μM and k_{cat}=25 s⁻¹.
h-FXIIa From “Enzyme Research Laboratories”. Product code FXIIa 1212A, lot #FXIIa 2520PL. Bottle with 0.5 mg was reconstituted with 370 μl pure water. Protein concentration: 1.35 mg/ml, i.e. ˜8 μM. Buffer 4 mM NaAc, 150 mM NaCl (pH 5.3). Preparation was divided into 20 μl amounts and frozen at −80° C.
Hepes735 140 mM NaCl, 20 mM Hepes, 0.02% NaN₃ (pH 7.35).
BSA5 Hepes735+5 mg/ml BSA.
CTI Prepared on 17 Dec. 2004, 0.141 mg/ml, i.e. 0.141/12028=11.7*10⁻⁶ M (11.7 μM). One portion was thawed and divided into 3 parts. Part 1 was frozen at −80° C.; part 2 was kept at RT; and part 3 was incubated 30 min at 95° C. and then kept at RT.

Effect of CTI, STI and TICA1 on Hydrolysis of S2302 by h-FXIIa

Stock FXIIa was diluted 700× in BSA5. The inhibitor was diluted in BSA5 as indicated. Wells contained 70 μl diluted FXIIa (5 nM final), 70 μl inhibitor and 20 μl S2302 (475 or 462.5 μM final). The reaction was started after 5 min heating on the heating plate with S2302. Filters 405 nm and 490 nm reference. Found mΔA/min was multiplied by 2.14 to get the figure for a 1 cm path.

The residual activity (V) was fitted against the PTI concentration with equation:

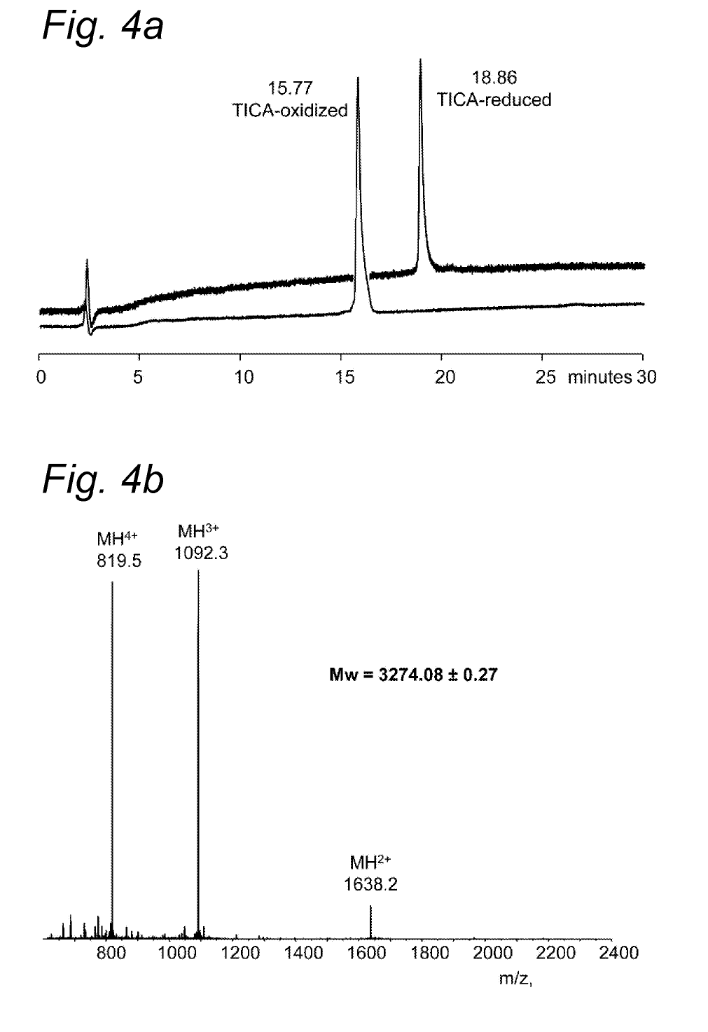

V=(E+(sqrt(Ê2+2*E*(Ki−I)+Î2+2*I*Ki+Kî2)−E−I−Ki)/2)*kcat*S/(Km+S)

in which E=[FXIIa], Ki is the dissociation constant, I=[Inhibitor], kcat and Km are Michaelis constants and S=[S2302]. FIG. 3 shows these fits and the derived constants for PTI and CTI. Table 4 summarizes the constants thus found.

TABLE 4
Dissociation constants of h-FXIIa - inhibitor complexes.

| Inhibitor | Dissociation constant (Ki) (nM) |
|---|---|
| CTI | 2.9 |
| STI | 251 |
| TICA1 | 25.5 |
| TICA2 | 109 |
| TICA3 (=HEE-TICA1) | 3,090 |
| TICA4 | 1,420 |
| TICA5 (=HEE-TICA4) | 171,000 |
| AAA-TICA1 | 104 |
| TICA1-AAA | 377 |
| PCTI | 469,000 |

### Example 4

### Chemical Synthesis of TICA1

General: 1. TICA1 was synthesised by solid phase peptide synthesis, cleaved from the resin (solid phase) and simultaneously deprotected, purified by HPLC, and lyophilized. 2. Purified TICA1 was dissolved, and oxidatively folded to obtain the disulfide bonded three-dimensional active structure. Final product was purifed by HPLC and lyophilised.

Specifically:

1. Synthesis: TICA1 polypeptide chain was synthesized by manual solid-phase peptide synthesis on a 0.2 mmol scale using the in situ neutralization/activation procedure for Boc-/Bzl-peptide synthesis as previously described, 10 but using HCTU instead of 2-(1H-benzotriazole-1-yl)-1,1,3,3-tetramethyluronium hexafluorophosphate (HBTU) as a coupling reagent. Glycyl PAM resin (0.76 meq/g) was used as the solid support. TICA1 peptide was deprotected and cleaved from the resin by treatment with anhydrous HF for 1 h at 0° C., using 4 v-% p-cresol as a scavenger. Following cleavage, TICA1 polypeptide chain was precipitated with ice-cold diethylether, dissolved in aqueous buffer containing 6 M Gn.HCl, 0.1 M sodium acetate buffer (pH 4) and purified by semi-preparative HPLC. Fractions containing the desired product were identified by ESI-MS, pooled and lyophilized.

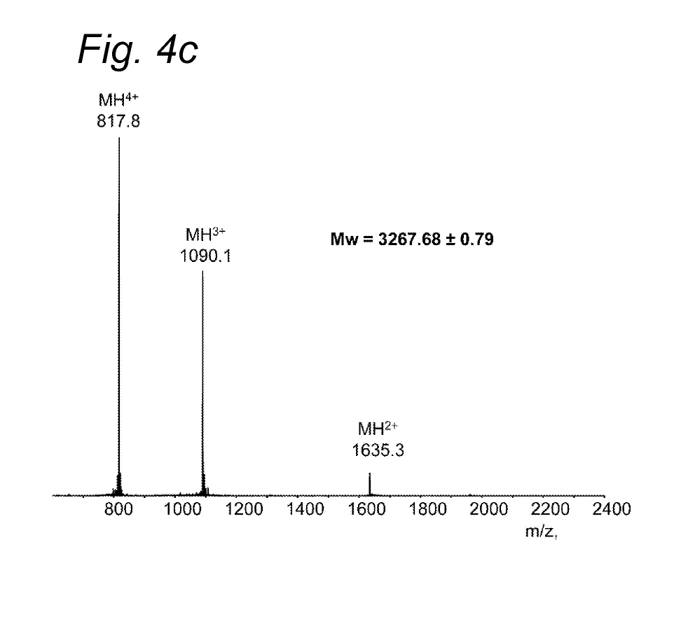

2. Oxidative Protein Folding: TICA1 (H-Arg1-Gly29-OH) (18 mg, 5.49 μmole) was dissolved in 50 mL of 1 M Gn.HCl, 0.10 M Tris buffer (pH 8.0) containing 8 mM cysteine and 1 mM cystine. The resulting solution was stirred for 2 h at 4° C. Reaction progress was monitored by analytical HPLC (FIG. 4). After semi-preparative HPLC, product containing fractions were identified by ESI-MS, pooled and lyophilized to give 10.05 mg of oxidized TICA1 (3.08 μmole, 56%). ESI-MS showed a mass of 3267.68+/−0.79, fitting well between the calculated monoisotopic mass (3266.50) and average mass (3268.99 of the folded TICA1. The observed mass difference of 6.4 Da between unfolded and folded TICA1 corresponds to the expected mass reduction caused by the loss of 6 protons due to the formation of 3 disulfide bonds (FIG. 4).

### Example 5

### Heat Stability of the Inhibitory Products

The following table shows the inhibitory activity of some of these products on the small molecular weight chromogenic peptide S2302, i.e. H-D-Pro-Phe-Arg-p.nitroaniline before and after heating, as well as the concentration that prolongs the clotting time of plasma that is optimally contact activated by preincubation with kaolin four times.

TABLE 5
Heat stability of inhibitory products.

| Inhibitor | Heating | TICA2 | TICA1 | TICA3 | TICA4 | TICA5 |
|---|---|---|---|---|---|---|
| kᵢ F.XIIa | Before | 355 | 83 | 10095 | 4621 | 556434 |
| (ng/ml) | | | | | | |
| kᵢ F.XIIa | After | | 85 | | | |
| (ng/ml) | | | | | | |
| kᵢ F.XIIa (nM) | Before | 109 | 25.5 | 3090 | 1420 | 171000 |
| kᵢ F.XIIa (nM) | After | | 26.1 | | | |
| 4 × lag-time | Before | 9.6 | 2.4 | ND | 31.6 | ND |
| (μM) | | | | | | |
| 4 × lag-time | After | 9.8 | 2.3 | ND | 31.4 | ND |
| (μM) | | | | | | |
| 4 × lag-time | Before | 31.1 | 7.8 | ND | 102.7 | ND |
| (ng/ml) | | | | | | |
| 4 × lag-time | After | 31.9 | 7.5 | ND | 102.9 | ND |
| (ng/ml) | | | | | | |

### Example 6

### Stability of CTI, PTI, and TICA at RT and 30 Min at 95° C.

Stock FXIIa was diluted 700× in BSA5. The inhibitor was diluted in BSA5 as indicated. Wells contained 70 μl diluted FXIIa (5 nM final), 70 μl inhibitor and 20 μl S2302 (475 or 462.5 μM final). The reaction was started after 5 min heating on the heating plate with S2302. Filters 405 nm and 490 nm reference. Found ΔmA/min was multiplied by 2.14 to get the figure for a 1 cm path.

The residual activity (V) was fitted against the inhibitor concentration with equation:

V=(E+(sqrt(Ê2+2*E*(Ki−I)+Î2+2*I*Ki+Kî2)−E−I−Ki)/2)*kcat*S/(Km+S)

in which E=[FXIIa], Ki is the dissociation constant, I=[Inhibitor], kcat and Km are Michaelis constants and S=[S2302].

TABLE 6
Effect of temperature of CTI, PTI and
TICA on its inhibition of h-FXIIa (5.01 nM)

| Inhibitor | Condition | (Ki) (nM) | Res. Act. (%) |
|---|---|---|---|
| CTI | −80° C. | 3.59 | 100 |
| | RT during 72 hrs | 4.19 | 86 |
| | 30 min 95° C. | 5.10 | 70 |
| PTI | −80° C. | 251 | 100 |
| | RT during 72 hrs | 267 | 106 |
| | 30 min 95° C. | 264 | 105 |
| TICA1 | −80° C. | 26 | 100 |
| | RT during 72 hrs | 26 | 102 |
| | 30 min 95° C. | 24 | 94 |
| TICA2 | −80° C. | 109 | 100 |
| | RT during 72 hrs | 114 | 105 |
| | 30 min 95° C. | 108 | 99 |
| TICA3 | −80° C. | 3090 | 100 |
| | RT during 72 hrs | 3128 | 101 |
| | 30 min 95° C. | 3180 | 103 |
| TICA4 | −80° C. | 1420 | 100 |
| | RT during 72 hrs | 1398 | 98 |
| | 30 min 95° C. | 1337 | 94 |
| TICA5 | −80° C. | 171000 | 100 |
| | RT during 72 hrs | 164531 | 96 |
| | 30 min 95° C. | 179243 | 105 |

Exposure of CTI and TICA1 for 60 minutes to 95° C.

To calculate the concentration of I the equation I=x·(x−E−Ki)/(x−E) was used. In this equation is I the calculated inhibitor concentration, x=the residual activity of FXIIa (free FXIIa), E the total FXIIa concentration (=9.33 nM), and Ki the dissociation or inhibitor constant.

| | Added | Activity of | Free | Calculated | Residual |
|---|---|---|---|---|---|
| | amount | FXIIa | FXIIa | total I | activity |
| Inhibitor | (nM) | (mA/min) | (nM) | (nM) | (%) |
| No Inhibitor | — | 79.6 ± 1.55 | 9.33 | — | 100 |
| CTI | 8.1 | 66.3 ± 1.32 | 7.77 | 2.37 | 29.3 |
| TICA1 | 306 | 33.5 ± 1.78 | 3.93 | 250 | 81.7 |

### Example 7

### Stability of TICA1 in Relation to Amount of Disulfide Bonds (0-1-2-3)

Chemical synthesis of TICA1 with 3 disulfide bonds is described in example 4 In order to synthesize TICA1 with less disulfide bonds, the synthesis and oxidative protein folding is different.

TICA1 with 2 Disulfide Bonds

Specifically:

1. Synthesis: TICA1 with 2 disulfide bonds was synthesized by manual solid-phase peptide synthesis as in example 4 except for the cystein residues. Two cystein residues responsible for a disulfide bond were replaced by alanine. Two other cystein residues responsible for a disulfide bond were replaced by cystein protected with acetamidomethyl (Acm) groups. The third cystein residues responsible for a disulfide bond were unchanged. The peptide was deprotected and cleaved as described in example 4. After HF cleavage, two cysteines are unprotected, the other two stayed protected with acetamidomethyl (Acm) groups. TICA1 polypeptide chain was precipitated with ice-cold diethylether, dissolved in aqueous buffer containing 50% acetonitril and 0.1% TFA, was analyzed by HPLC and lyophilized.

2. Oxidative Protein Folding: In order to generate the first disulfide bond, TICA1 was dissolved in 1 M Gn.HCl, 0.05 M Tris buffer (pH 8.0) (0.5 mg/ml). The resulting solution was stirred for 48 h at 4° C. Then for the formation of the second disulfide bond the solution was adjusted to 10% AcOH, purged with nitrogen, and the Acm groups were removed by addition of 2 equivalents Iodine (0.12M in methanol). Reaction progress was monitored by analytical HPLC and ESI-MS, showing a mass fitting well between the calculated monoisotopic mass and average mass of the folded peptide. After semi-preparative HPLC, product containing fractions were identified by ESI-MS, pooled and lyophilized. The observed mass difference of −144.2 Da corresponds to the expected mass reduction caused by the loss of 2 protons and 2 Acm groups due to the formation of 2 disulfide bonds.

TICA1 with 1 Disulfide Bond

Specifically:

1. Synthesis: TICA1 with 2 disulfide bonds was synthesized by manual solid-phase peptide synthesis as in example 4 exept for the cystein residues. Four cystein residues responsible for 2 disulfide bonds were replaced by alanine. The peptide was deprotected and cleaved as described in example 4. Following cleavage, TICA1 polypeptide chain was precipitated with ice-cold diethylether, dissolved in aqueous buffer containing 6 M Gn.HCl, 0.1 M sodium acetate buffer (pH 4) and purified by semi-preparative HPLC. Fractions containing the desired product were identified by ESI-MS, pooled and lyophilized.

2. Oxidative Protein Folding: Oxidative Protein Folding: TICA1 was dissolved in 1 M Gn.HCl, 0.10 M Tris buffer (pH 8.0) The resulting solution was stirred for 24 h at 4° C. Reaction progress was monitored by analytical HPLC (FIG. 4). After semi-preparative HPLC, product containing fractions were identified by ESI-MS, pooled and lyophilized. ESI-MS showed an observed mass difference of 2 Da between unfolded and folded TICA1 corresponds to the expected mass reduction caused by the loss of 2 protons due to the formation of 1 disulfide bond (FIG. 4).

TICA1 without Disulfide Bonds

Specifically:

1. Synthesis: TICA1 was synthesized by manual solid-phase peptide synthesis as in example 4. The peptide was deprotected and cleaved as described in example 4. Following cleavage, reduced TICA1 polypeptide chain was precipitated with ice-cold diethylether, dissolved in aqueous buffer containing 6 M Gn.HCl, 0.1 M sodium acetate buffer (pH 4) and purified by semi-preparative HPLC. Fractions containing the desired product were identified by ESI-MS, pooled and lyophilized.

2. Blocking Cysteines (“Ac” in example 7): reduced TICA1 was dissolved in 50 mM ammonium bicarbonate pH 7.8 (1 mg/ml). 40 mM Iodoacetamide (7.4 mg/ml) was added. The reaction took place for 1 hour in the dark. Reaction progress was monitored by analytical HPLC After semi-preparative HPLC, product containing fractions were identified by ESI-MS, pooled and lyophilized. ESI-MS showed an observed mass difference of 342 Da between unblocked and blocked cysteins corresponds to the expected mass increase caused by adding 6 acetamides to the cysteines.

### Example 8

### Ala-Scan with Intact Disulfides

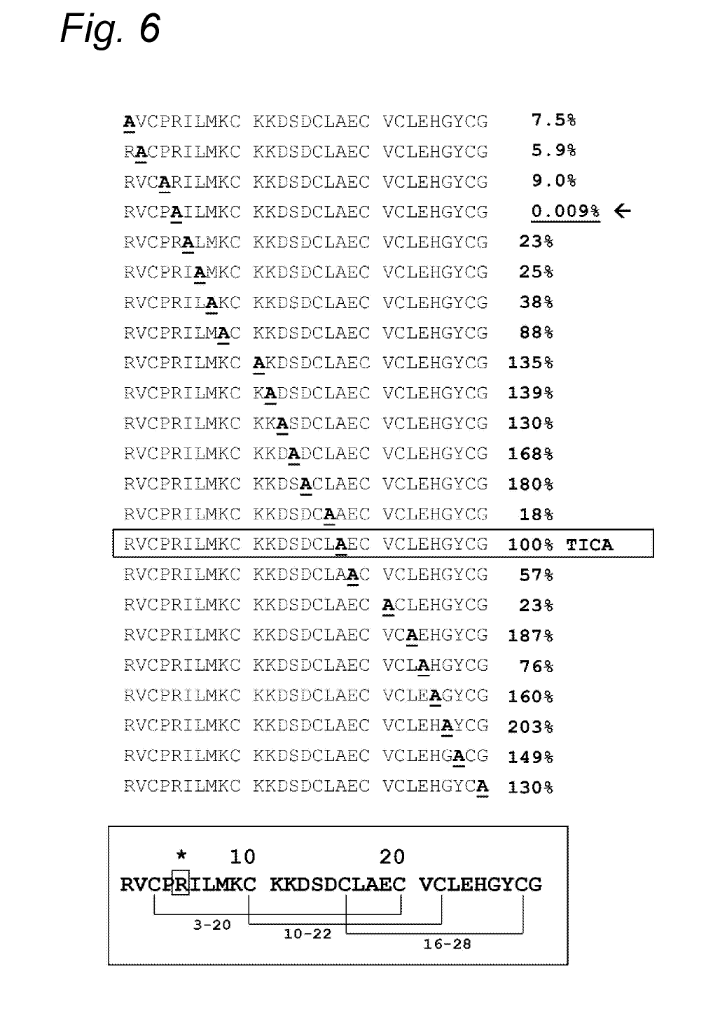

The contribution of every amino acid to the activity of TICA1 was determined. Every single amino acid was substituted by alanine except for cysteine. The effect of cysteine in TICA1 was already described in example 7. All TICA1-analoges were synthesized and folded as described in example 4. See also FIG. 6.

| | Activity |
|---|---|
| Sequence Ala scan | vs TICA1 |
| AVCPRILMKCKKDSDCLAECVCLEHGYCG | 7.5% |
| RACPRILMKCKKDSDCLAECVCLEHGYCG | 5.9% |
| RVCARILMKCKKDSDCLAECVCLEHGYCG | 9.0% |
| RVCPAILMKCKKDSDCLAECVCLEHGYCG | 0.009% |
| RVCPRALMKCKKDSDCLAECVCLEHGYCG | 23% |
| RVCPRIAMKCKKDSDCLAECVCLEHGYCG | 25% |
| RVCPRILAKCKKDSDCLAECVCLEHGYCG | 38% |
| RVCPRILMACKKDSDCLAECVCLEHGYCG | 88% |
| RVCPRILMKCAKDSDCLAECVCLEHGYCG | 135% |
| RVCPRILMKCKADSDCLAECVCLEHGYCG | 139% |
| RVCPRILMKCKKASDCLAECVCLEHGYCG | 130% |
| RVCPRILMKCKKDADCLAECVCLEHGYCG | 168% |
| RVCPRILMKCKKDSACLAECVCLEHGYCG | 180% |
| RVCPRILMKCKKDSDCAAECVCLEHGYCG | 18% |
| RVCPRILMKCKKDSDCLAECVCLEHGYCG | 100% |
| | (=TICA1) |
| RVCPRILMKCKKDSDCLAACVCLEHGYCG | 57% |
| RVCPRILMKCKKDSDCLAECACLEHGYCG | 23% |
| RVCPRILMKCKKDSDCLAECVCAEHGYCG | 187% |
| RVCPRILMKCKKDSDCLAECVCLAHGYCG | 76% |
| RVCPRILMKCKKDSDCLAECVCLEAGYCG | 160% |
| RVCPRILMKCKKDSDCLAECVCLEHAYCG | 203% |
| RVCPRILMKCKKDSDCLAECVCLEHGACG | 149% |
| RVCPRILMKCKKDSDCLAECVCLEHGYCA | 130% |

### Example 9

### The Effect of TICA Addition to Blood Collection Tubes and CAT Assay

In 12 donors blood was drawn with 7 different drawing tubes with and without addition of TICA1 in the drawing tubes (total 14 tubes; 78 ml). After preparing PPP from these tubes, CAT measurements with MP reagent and pplow reagent was performed. One donor is measured in one plate. (pplow duplicate; MP duplicate; calibrator in 4 fold. Total volume is 320 μl sample/measurement in total 88 wells/donor.

In 12 donors blood is taken with the following commercial available drawing tubes: (From all blooddrawing tubes one tube with and one tube without TICA will be used to draw blood)

Start blooddrawing with one edta tube.

1A: BD vacutainer (glass) 9NC; 0.5 ml 0.105M citrate, total volume 5 ml.

1C: Add to one vacuum tube: 100 μl, 1.5 mg/ml TICA

Total tubes: 2=>one tube with TICA and one tube without TICA.

2A: Sarstedt tubes; S-monovette, 9NC; 0.5 ml 0.106M citrate, total volume 5 ml.

2C: Add to one vacuum tube: 100 μl, 1.5 mg/ml TICA

Total tubes: 2=>one tube with TICA and one tube without TICA.

3A: Terumo Venosafe 4 ml. 9NC; 0.4 ml 0.109M citrate, total volume 4 ml. ppg internal coating/silicone stopper coating

3C: Add to one vacuum tube: 80 μl, 1.5 mg/ml TICA.

Total tubes: 2=>one tube with TICA and one tube without TICA.

4A: BD Vacutainer, (plastic) 9NC; 0.3 ml 0.109M citrate, total volume 3 ml.

4C: Add to one vacuum tube: 60 μl, 1.5 mg/ml TICA.

Total tubes: 2=>one tube with TICA and one tube without TICA.

5A: Haemtech SCAT 113-4.5/5 AA0809 3.2% citrate. (Control tubes without CTI), total volume 5 ml.

5C: Add to one vacuum tube: 100 μl, 1.5 mg/ml TICA.

Total tubes: 2=>one tube with TICA and one tube without TICA.

6A: Greiner vacuum tubes 9 ml+1 ml (9NC, 3.2% citrate)

6C: Add to one vacuum tube: 200 μl, 1.5 mg/ml TICA.

Total tubes: 2=>one tube with TICA and one tube without TICA.

Final TICA concentration after blood drawing in every tube with added TICA is 30 μg/ml whole blood.

Final TICA concentration in plasma after preparing PPP is 60 μg/ml (assuming an hematocit of 50%)

TICA Preparation:

Dissolve 9 mg TICA in 6 ml sterile injection water. This solution is used for injection of TICA in the drawing tube. After injection of TICA solution in the blood drawing tube this tube has to be used within 8 hours.

CAT

Measurement No TICA and TICA Added to Tube:

CAT measurements with MP reagent and pplow reagent will be performed. One donor is measured in one plate. PPLow duplicate; MP duplicate; calibrator in 4 fold.

Total volume is 320 μl sample/measurement in total 88 wells/donor. NP2012 is also included in the measurement in 4 fold.

Lag times, Endogenous thrombin potentials, and peak height were recorded, For the current conditions of thrombin generation, peak height is the preferred parameter for interpretation of the results.

Measurement TICA Added to CAT Assay:

Plasma with TICA added: concentration in plasma: 60 μg/ml

Stock TICA: 1.5 mg/ml=>1500 μg/ml

20 μl TICA (stock: 1500 μg/ml) is added to 480 μl plasma.

| | | TICA added | TICA added |
|---|---|---|---|
| TICA series in 9 volunteers | No Tica | to CAT assay | to Tube |
| BD Glass 0 pM TF Lagtime | 12.8 ± 3.2 | 38.8 ± 34.8 | 76.6 ± 34.0 |
| (min) | | | |
| BD Glass 0 pM TF ETP | 1053 ± 334 | 760 ± 526 | 77 ± 119 |
| (nM IIa · min) | | | |
| BD Glass 0 pM TF Peak | 165 ± 61 | 105 ± 81 | 6 ± 9 |
| Height (nM IIa) | | | |
| BD Plastic 0 pM TF | 14.8 ± 6.5 | 49.5 ± 37.7 | 90.1 ± 26.8 |
| Lagtime (min) | | | |
| BD Plastic 0 pM TF ETP | 910 ± 472 | 614 ± 538 | 20 ± 61 |
| (nM IIa · min) | | | |
| BD Plastic 0 pM TF Peak | 162 ± 71 | 86 ± 87 | 2 ± 6 |
| Height (nM IIa) | | | |
| Sarstedt 0 pM TF Lagtime | 16.7 ± 16.9 | 50.0 ± 38.0 | 52.5 ± 36.2 |
| (min) | | | |
| Sarstedt 0 pM TF ETP | 921 ± 445 | 279 ± 227 | 140 ± 106 |
| (nM IIa · min) | | | |
| Sarstedt 0 pM TF Peak | 126 ± 70 | 26 ± 23 | 10 ± 8 |
| Height (nM IIa) | | | |
| Terumo 0 pM TF Lagtime | 26.1 ± 29.9 | 64 ± 40.6 | 75.7 ± 35.6 |
| (min) | | | |
| Terumo 0 pM TF ETP | 846 ± 523 | 276 ± 367 | 59 ± 119 |
| (nM IIa · min) | | | |
| Terumo 0 pM TF Peak | 117 ± 95 | 33 ± 50 | 5 ± 11 |
| Height (nM IIa) | | | |
| Greiner 0 pM TF Lagtime | 13.1 ± 3.0 | 31.2 ± 26.7 | 65.0 ± 34.8 |
| (min) | | | |
| Greiner 0 pM TF ETP | 992 ± 313 | 834 ± 449 | 144 ± 164 |
| (nM IIa · min) | | | |
| Greiner 0 pM TF Peak | 147 ± 62 | 110 ± 70 | 11 ± 13 |
| Height (nM IIa) | | | |
| Heamtech 0 pM TF | 21.7 ± 29.2 | 46.4 ± 39.7 | 65.0 ± 34.8 |
| Lagtime (min) | | | |
| Heamtech 0 pM TF ETP | 981 ± 521 | 666 ± 552 | 289 ± 420 |
| (nM IIa · min) | | | |
| Heamtech 0 pM TF Peak | 155 ± 90 | 93 ± 85 | 36 ± 62 |
| Height (nM IIa) | | | |

### REFERENCE LIST

1. U.S. Pat. No. 6,403,381.
2. WO2010/016762
3. WO2003/093831
4. WO2006/117246
5. “The blood coagulation cascade” by Schenone M, Furie BC and Furie B. Current Opinion in Haematology 2004, Vol 11(4) pages 272-277.
6. L. Poller in “Laboratory techniques in thrombosis” Edited by J. Jespersen, R. M. Bertina and F. Haverkate, ISBN 0-7923-5317-X (1999) p. 37-44.
7. “Laboratory techniques in thrombosis” Edited by J. Jespersen, R. M. Bertina and F. Haverkate, ISBN 0-7923-5317-X (1999);
8. “Haemostasis and thrombosis: Basic principles and clinical practice” Eds: Colman, Hirsh, Marder, Clowes, George Lippincott Williams and Wilkins. (2010).

1. A composition comprising at least one calcium-chelating substance with blood clotting inhibition activity and further comprising a thermostable peptide of at most 42 amino acids which inhibits contact activation of the blood clotting system, said peptide comprising within a consecutive sequence of at most about 40 amino acids at least four cysteines, the amino acid Arg or an analog of Arg on a position corresponding to amino acid position 5 in SEQ ID NO: 1 and preferably at least the consecutive amino acid sequence Ile-Leu-Met.

2. A device for collecting a sample comprising the composition of claim 1.

3. A kit of parts comprising:
the device of claim 2, and
a reagent for determining blood clotting activity, preferably the development of thrombin activity as a function of time.

4. Use of a thermostable peptide of at most 42 amino acids which inhibits contact activation of the blood clotting system, said peptide comprising within a consecutive sequence of at most about 40 amino acids at least four cysteines and preferably at least the consecutive amino acid sequence Ile-Leu-Met, for inhibiting activation of the blood clotting system through contact with foreign surfaces.

5. A thermostable peptide of at most 42 amino acids which inhibits contact activation of the blood clotting system, said peptide comprising within a consecutive sequence of at most about 40 amino acids at least four cysteines, the amino acid Arg or an analog of Arg on a position corresponding to amino acid position 5 in SEQ ID NO: 1 and preferably at least the consecutive amino acid sequence Ile-Leu-Met.

6. A method for determining blood clotting activity, said method comprising:
collecting a blood sample from an individual, wherein the thermostable peptide of claim 5 is contacted with the blood sample,
measuring in said sample blood clotting activity, preferably thrombin activity, more preferably using a fluorescent assay for measuring thrombin activity, and preferably,
using the blood clotting activity value for diagnosis.

7. A method for inhibiting clotting of blood, the method comprising contacting a thermostable peptide of claim 5, with blood, plasma or any other biological fluid in an amount sufficient to inhibit the clotting.

8. A method for collection and preparation of a sample from an individual comprising:
providing a device for collecting a sample, said device comprising at least one calcium-chelating substance with blood clotting inhibition activity and further comprising a thermostable peptide of claim 5,
collecting a sample into the device, and
mixing sample, calcium-chelating substance with blood clotting inhibition activity and peptide.

9. A method for determining blood clotting activity in a blood sample, the method comprising: measuring in a blood sample, plasma sample or any other biological fluid comprising at least one calcium chelating substance with blood clotting inhibition activity and further comprising a thermostable peptide of claim 5, blood clotting activity, preferably thrombin activity, more preferably using a fluorescent assay for measuring thrombin activity.

10. (canceled)

11. (canceled)

12. A composition according to claim 1 wherein the thermostable peptide which inhibits contact activation of the blood clotting system, comprises a consensus amino acid sequence: XXCXXXXXXCXXXXXCXXXCXCXXXXXCX (SEQ ID NO: 9), wherein X at position 5 is R or an analog of R; X at position 9 is E or K, preferably K and X at position 21 is I or V, preferably V, or more preferably: XILMXCKKDSDCLAECX (SEQ ID NO: 6), wherein X at position 1 is K or R or an analog of R, preferably R or an analog of R; X at position 5 is E or K, preferably K and X at position 17 is I or V, preferably V or more preferably: RVCPXILMXCKKDSDCLAECXCLEHGYCG (SEQ ID NO: 7), wherein X at position 5 is K or R or an analog of R, preferably R or an analog of R; X at position 9 is E or K, preferably K and X at position 21 is I or V, preferably V.

13. A composition according to claim 1 wherein the thermostable peptide which inhibits contact activation of the blood clotting system, comprises a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a sequence as shown in SEQ ID NO: 1, SEQ ID NO: 2, SEQ ID NO: 3, or SEQ ID NO: 4.

14. A composition according to claim 1 wherein the thermostable peptide which inhibits contact activation of the blood clotting system, is a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a peptide as shown in SEQ ID NO: 1, SEQ ID NO: 2, SEQ ID NO: 3, or SEQ ID NO: 4.

15. A polynucleotide comprising a polynucleotide encoding a thermostable peptide as described in claim 5.

16. A medicament comprising the thermostable peptide of claim 5.

17. A device according to claim 2 wherein the thermostable peptide which inhibits contact activation of the blood clotting system, comprises a consensus amino acid sequence: XXCXXXXXXCXXXXXCXXXCXCXXXXXCX (SEQ ID NO: 9), wherein X at position 5 is R or an analog of R; X at position 9 is E or K, preferably K and X at position 21 is I or V, preferably V, or more preferably: XILMXCKKDSDCLAECX (SEQ ID NO: 6), wherein X at position 1 is K or R or an analog of R, preferably R or an analog of R; X at position 5 is E or K, preferably K and X at position 17 is I or V, preferably V or more preferably: RVCPXILMXCKKDSDCLAECXCLEHGYCG (SEQ ID NO: 7), wherein X at position 5 is K or R or an analog of R, preferably R or an analog of R; X at position 9 is E or K, preferably K and X at position 21 is I or V, preferably V.

18. A use according to claim 4 wherein the thermostable peptide which inhibits contact activation of the blood clotting system, comprises a consensus amino acid sequence: XXCXXXXXXCXXXXXCXXXCXCXXXXXCX (SEQ ID NO: 9), wherein X at position 5 is R or an analog of R; X at position 9 is E or K, preferably K and X at position 21 is I or V, preferably V, or more preferably: XILMXCKKDSDCLAECX (SEQ ID NO: 6), wherein X at position 1 is K or R or an analog of R, preferably R or an analog of R; X at position 5 is E or K, preferably K and X at position 17 is I or V, preferably V or more preferably: RVCPXILMXCKKDSDCLAECXCLEHGYCG (SEQ ID NO: 7), wherein X at position 5 is K or R or an analog of R, preferably R or an analog of R; X at position 9 is E or K, preferably K and X at position 21 is I or V, preferably V.

19. A peptide according to claim 5 wherein the thermostable peptide which inhibits contact activation of the blood clotting system, comprises a consensus amino acid sequence: XXCXXXXXXCXXXXXCXXXCXCXXXXXCX (SEQ ID NO: 9), wherein X at position 5 is R or an analog of R; X at position 9 is E or K, preferably K and X at position 21 is I or V, preferably V, or more preferably: XILMXCKKDSDCLAECX (SEQ ID NO: 6), wherein X at position 1 is K or R or an analog of R, preferably R or an analog of R; X at position 5 is E or K, preferably K and X at position 17 is I or V, preferably V or more preferably: RVCPXILMXCKKDSDCLAECXCLEHGYCG (SEQ ID NO: 7), wherein X at position 5 is K or R or an analog of R, preferably R or an analog of R; X at position 9 is E or K, preferably K and X at position 21 is I or V, preferably V.

20. A device according to claim 2 wherein the thermostable peptide which inhibits contact activation of the blood clotting system, comprises a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a sequence as shown in SEQ ID NO: 1, SEQ ID NO: 2, SEQ ID NO: 3, or SEQ ID NO: 4.

21. A use according to claim 4 wherein the thermostable peptide which inhibits contact activation of the blood clotting system, comprises a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a sequence as shown in SEQ ID NO: 1, SEQ ID NO: 2, SEQ ID NO: 3, or SEQ ID NO: 4.

22. A peptide according to claim 5 wherein the thermostable peptide which inhibits contact activation of the blood clotting system, comprises a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a sequence as shown in SEQ ID NO: 1, SEQ ID NO: 2, SEQ ID NO: 3, or SEQ ID NO: 4.

23. A device according to claim 2 wherein the thermostable peptide which inhibits contact activation of the blood clotting system, is a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a peptide as shown in SEQ ID NO: 1, SEQ ID NO: 2, SEQ ID NO: 3, or SEQ ID NO: 4.

24. A use according to claim 4 wherein the thermostable peptide which inhibits contact activation of the blood clotting system, is a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a peptide as shown in SEQ ID NO: 1, SEQ ID NO: 2, SEQ ID NO: 3, or SEQ ID NO: 4.

25. A peptide according to claim 5 wherein the thermostable peptide which inhibits contact activation of the blood clotting system, is a peptide selected from the group consisting of:
a peptide with the sequence as shown in SEQ ID NO: 1,
a peptide with the sequence as shown in SEQ ID NO: 2,
a peptide with the sequence as shown in SEQ ID NO: 3,
a peptide with the sequence as shown in SEQ ID NO: 4, and
a peptide which has at least 70% sequence identity with a peptide as shown in SEQ ID NO: 1, SEQ ID NO: 2, SEQ ID NO: 3, or SEQ ID NO: 4.


SEQUENCE LISTING (25 sequences)

SEQ ID NO: 1 - protein, 29 aa, Artificial
  Peptide
RVCPRILMKCKKDSDCLAECVCLEHGYCG

SEQ ID NO: 2 - protein, 29 aa, Artificial
  Peptide
RVCPRILMECKKDSDCLAECVCLEHGYCG

SEQ ID NO: 3 - protein, 32 aa, Artificial
  Peptide
HEERVCPRILMKCKKDSDCLAECVCLEHGYCG

SEQ ID NO: 4 - protein, 29 aa, Artificial
  Peptide
RVCPKILMECKKDSDCLAECICLEHGYCG

SEQ ID NO: 5 - protein, 32 aa, Artificial
  Peptide
HEERVCPKILMECKKDSDCLAECICLEHGYCG

SEQ ID NO: 6 - protein, 17 aa, Artificial
  Peptide
XILMXCKKDSDCLAECX

SEQ ID NO: 7 - protein, 29 aa, Artificial
  Peptide
RVCPXILMXCKKDSDCLAECXCLEHGYCG

SEQ ID NO: 8 - protein, 39 aa, Artificial
  Peptide
RVCGIGPRPRLPWPRILMKCKKDSDCLAECVCLEHGYCG

SEQ ID NO: 9 - protein, 29 aa, Artificial
  Peptide
XXCXXXXXXCXXXXXCXXXCXCXXXXXCX

SEQ ID NO: 10 - protein, 29 aa, Artificial
  Peptide
RXCXXXXXXCXXXXXCXXXCXCXXXXXCX

SEQ ID NO: 11 - protein, 29 aa, Artificial
  Peptide
XVCXXXXXXCXXXXXCXXXCXCXXXXXCX

SEQ ID NO: 12 - protein, 29 aa, Artificial
  Peptide
XXCPXXXXXCXXXXXCXXXCXCXXXXXCX

SEQ ID NO: 13 - protein, 29 aa, Artificial
  Peptide
XXCXRXXXXCXXXXXCXXXCXCXXXXXCX

SEQ ID NO: 14 - protein, 29 aa, Artificial
  Peptide
RVCXXXXXXCXXXXXCXXXCXCXXXXXCX

SEQ ID NO: 15 - protein, 29 aa, Artificial
  Peptide
RXCPXXXXXCXXXXXCXXXCXCXXXXXCX

SEQ ID NO: 16 - protein, 29 aa, Artificial
  Peptide
RXCXRXXXXCXXXXXCXXXCXCXXXXXCX

SEQ ID NO: 17 - protein, 29 aa, Artificial
  Peptide
XVCPXXXXXCXXXXXCXXXCXCXXXXXCX

SEQ ID NO: 18 - protein, 29 aa, Artificial
  Peptide
XVCXRXXXXCXXXXXCXXXCXCXXXXXCX

SEQ ID NO: 19 - protein, 29 aa, Artificial
  Peptide
XXCPRXXXXCXXXXXCXXXCXCXXXXXCX

SEQ ID NO: 20 - protein, 29 aa, Artificial
  Peptide
RXCXRXXXXCXXXXXCXXXCXCXXXXXCX

SEQ ID NO: 21 - protein, 29 aa, Artificial
  Peptide
RVCPXXXXXCXXXXXCXXXCXCXXXXXCX

SEQ ID NO: 22 - protein, 29 aa, Artificial
  Peptide
RVCXRXXXXCXXXXXCXXXCXCXXXXXCX

SEQ ID NO: 23 - protein, 29 aa, Artificial
  Peptide
XVCPRXXXXCXXXXXCXXXCXCXXXXXCX

SEQ ID NO: 24 - protein, 29 aa, Artificial
  Peptide
RXCPRXXXXCXXXXXCXXXCXCXXXXXCX

SEQ ID NO: 25 - protein, 29 aa, Artificial
  Peptide
RVCPRXXXXCXXXXXCXXXCXCXXXXXCX